(19) United States
(12) Patent Application Publication
(10) Pub. No.: US 2005/0232987 A1
(43) Pub. Date: Oct. 20, 2005

(54) Dosage form containing a morphine derivative and another drug

(76) Inventors: Viswanathan Srinivasan, The Woodlands, TX (US); Ralph Brown, South Lake, TX (US); David Brown, Colleyville, TX (US); Himanshu Patel, North Richland, TX (US); Juan Carlos Menendez, Bedford, TX (US); Venkatesh Balasubramanian, Arlington, TX (US)

Correspondence Address: GREENBLUM & BERNSTEIN, P.L.C., 1950 ROLAND CLARKE PLACE, RESTON, VA, 20191, US

(21) Appl. No.: 10/798,884

(22) Filed: Mar. 12, 2004

(51) Int. Cl. A61K 31/485; A61K 31/137; A61K 9/00; A61K 9/20

(52) U.S. Cl. 424/464; 514/282; 514/649

(57) ABSTRACT

A pharmaceutical dosage form which comprises a first drug which comprises at least one morphine derivative with antitussive activity and at least one second drug. The dosage form provides a plasma concentration within the therapeutic range of the at least one second drug over a period which is coextensive with at least about 70% of the period over which the dosage form provides a plasma concentration within the therapeutic range of the first drug. This abstract is neither intended to define the invention disclosed in this specification nor intended to limit the scope of the invention in any way.

### FIELD OF THE INVENTION

The present invention relates to a pharmaceutical dosage form which contains a morphine derivative with antitussive activity such as, e.g., codeine, dihydrocodeine, hydrocodone and/or a pharmaceutically acceptable salt thereof in combination with at least one additional active ingredient. The dosage form releases the morphine derivative and the at least one additional active ingredient at rates which provide pharmaceutically suitable plasma concentrations thereof over similar periods of time. The present invention also relates to a process for manufacturing the dosage form and to methods for alleviating excessive coughing in a patient by administering the dosage form to the patient.

### DISCUSSION OF BACKGROUND INFORMATION

Morphine derivatives such as codeine, dihydrocodeine and hydrocodone possess antitussive and pain relieving properties. The dosages sufficient for ameliorating excessive coughing by taking advantage of the antitussive properties of these narcotic agents are usually lower than the dosages needed to ameliorate pain. Furthermore, the agents needed in combination with these narcotic agents for the relief of excessive coughing are usually different from those required as adjuncts in the treatment of pain. Excessive coughing, which can be treated or ameliorated with a morphine derivative such as codeine, dihydrocodeine and hydrocodone, is often accompanied by conditions which cannot satisfactorily be ameliorated or treated with the morphine derivative, but may be treated or ameliorated by other drugs such as, e.g., expectorants, mucus thinning drugs, decongestants and/or antihistamines. However, a single pharmacologically acceptable dose (i.e., a dose which will not result in a plasma concentration which causes unacceptable side-effects) of, for example, codeine, dihydrocodeine and/or hydrocodone provides a therapeutically effective plasma concentration for 2.5±0.7 hours whereas many agents frequently used in conjunction with these morphine derivatives provide therapeutically effective plasma concentrations per single pharmacologically acceptable dose over periods that differ markedly from that provided by these morphine derivatives. For example, a single pharmacologically acceptable dose of an expectorant such as guaifenesin will usually provide relief for about one hour, and decongestants usually provide relief for about 4 to 8 hours per single dose. As a result, there appears to be virtually no benefit in combining a morphine derivative such as, e.g., codeine, dihydrocodeine and/or hydrocodone and any such drug with a noticeably shorter or longer therapeutically effective period in a single dosage form. With a corresponding combination, one drug (e.g., the morphine derivative) may still provide the desired therapeutic effect when the other drug has already ceased to be effective, or the other drug may continue to exert a therapeutic effect, which prohibits administration of another dose thereof even though the morphine derivative no longer provides the desired antitussive effect.

It would be desirable if patients suffering from, e.g., excessive coughing, respiratory congestion, inflammation of the respiratory mucosa and sinus cavities, weeping eyes, rhinorrhea, Eustachian Tube congestion, nausea and related symptoms, for which a morphine derivative such as, e.g., codeine, dihydrocodeine and hydrocodone is indicated, would also obtain relief, over a similar time period, from one or more conditions for which drugs different from the morphine derivative are indicated, by administering a single dose of a dosage form such as, e.g., a tablet, liquid, syrup, suspension, capsule and the like which contains both the morphine derivative and one or more other drugs.

### SUMMARY OF THE INVENTION

The present invention provides a pharmaceutical dosage form which comprises a first drug which comprises at least one morphine derivative with antitussive activity and at least one second drug. This dosage form provides a plasma concentration within the therapeutic range of the at least one second drug over a period which is coextensive with at least about 70% of the period over which the dosage form provides a plasma concentration within the therapeutic range of the first drug.

In one aspect of the dosage form, the at least one morphine derivative may comprise codeine and/or dihydrocodeine and/or hydrocodone and/or one or more pharmaceutically acceptable salts thereof. For example, the first drug may comprise codeine phosphate, dihydrocodeine bitartrate and/or hydrocodone bitartrate. Preferably, it comprises at least codeine phosphate.

In another aspect, the at least one second drug may comprise a decongestant and/or an expectorant and/or a mucus thinning drug and/or an antihistamine. By way of non-limiting example, the at least one second drug may comprise a decongestant, for example, phenylepherine and/or pseudoephedrine and/or one or more pharmaceutically acceptable salts thereof; and/or the at least one second drug may comprise an antihistamine, for example, chlorpheniramine and/or promethazine and/or carbinoxamine and/or controlled release layer(s) of a multi-layered (e.g., bi-layered) tablet one or more pharmaceutically acceptable salts thereof, and/or the at least one second drug may comprise an expectorant, for example, guaifenesin.

In yet another aspect of the dosage form, the plasma half-life of the at least one second drug may differ from the plasma half-life of the first drug (i.e., may be longer or may be shorter) by at least about 2 hours, e.g., by at least about 3 hours, or by at least about 4 hours.

In a still further aspect, the period of a plasma concentration within the therapeutic range of the at least one second drug may be coextensive with at least about 80%, e.g., at least about 90%, or even at least about 95%, of the period within which the plasma concentration of the first drug is within the therapeutic range.

In another aspect, the dosage form may be a tablet. This tablet may have at least two layers. It may, for example, be a bi-layered tablet. In another embodiment, the tablet may comprise a matrix which comprises the first drug and has dispersed therein particles which comprise the at least one second drug.

In yet another aspect, the dosage form may be a liquid and may comprise a solution and/or a suspension.

The present invention also provides a bi-layered tablet having a first layer and a second layer. The first layer comprises a first drug which comprises at least one morphine derivative with antitussive activity, and the second layer comprises at least one second drug which is selected from decongestants, expectorants, mucus thinning drugs, and antihistamines. This bi-layered tablet provides a plasma concentration within the therapeutic range of the at least one second drug over a period which is coextensive with at least about 70% of the period over which the bi-layered tablet provides a plasma concentration within the therapeutic range of the first drug.

In one aspect of the bi-layered tablet, the first layer may comprise codeine, dihydrocodeine, hydrocodone and/or one or more pharmaceutically acceptable salts thereof.

In another aspect of the bi-layered tablet of the present invention, the second layer thereof may comprise one or more of codeine, dihydrocodeine, hydrocodone, phenylepherine, pseudoephedrine, chlorpheniramine, carbinoxamine, promethazine, guaifenesin and pharmaceutically acceptable salts thereof.

In another aspect, the tablet may comprise at least two of codeine, dihydrocodeine, hydrocodone, phenylepherine, pseudoephedrine, chlorpheniramine, carbinoxamine, promethazine, guaifenesin and pharmaceutically acceptable salts thereof (contained in only the second layer or in both the first layer and the second layer).

In a still further aspect, the first layer of the bi-layered tablet may comprise one or more of codeine, dihydrocodeine, hydrocodone and pharmaceutically acceptable salts thereof as the only active ingredient(s).

In yet another aspect of the tablet, the period of a plasma concentration within the therapeutic range of the at least one second drug which is provided by the tablet may be coextensive with at least about 80%, preferably at least about 90%, of the period over which the tablet provides a plasma concentration within the therapeutic range of the first drug.

In another aspect of the tablet, the first layer and/or the second layer thereof may be an immediate release layer. For example, the first layer may be an immediate release layer, or the second layer may be an immediate release layer.

In yet another aspect, both of the first and second layers may be controlled release layers (which may provide different release rates and/or may exhibit different times at which the release of the active ingredient(s) starts, etc.).

In a still further aspect of the tablet, the first layer may comprise a total of from about 0.1 mg to about 120 mg, e.g., from about 5 mg to about 90 mg, or from about 25 mg to about 50 mg of codeine and/or dihydrocodeine and/or hydrocodone and/or one or more pharmaceutically acceptable salts thereof; and/or the second layer may comprise (i) from about 0.1 mg to about 16 mg of chlorpheniramine maleate or an equivalent amount of at least one other pharmaceutically acceptable salt of chlorpheniramine; and/or (ii) from about 1 mg to about 90 mg of phenylepherine hydrochloride or an equivalent amount of at least one other pharmaceutically acceptable salt of phenylepherine; and/or (iii) from about 1 mg to about 240 mg of pseudoephedrine hydrochloride or an equivalent amount of at least one other pharmaceutically acceptable salt of pseudoephedrine; and/or (iv) from about 0.1 mg to about 75 mg of promethazine hydrochloride or an equivalent amount of at least one other pharmaceutically acceptable salt of promethazine; and/or (v) from about 0.1 mg to about 32 mg of carbinoxamine maleate or an equivalent amount of at least one other pharmaceutically acceptable salt of carbinoxamine; and/or (vi) form about 1 mg to about 2400 mg of guaifenesin or an equivalent amount of at least one pharmaceutically acceptable salt of guaifenesin.

In another aspect of the bi-layered tablet, the first layer may comprise, in addition to the at least one morphine derivative having antitussive activity, (i) from about 1 mg to about 90 mg of phenylepherine hydrochloride or an equivalent amount of at least one other pharmaceutically acceptable salt of phenylepherine; and/or (ii) from about 1 mg to about 240 mg of pseudoephedrine hydrochloride or an equivalent amount of at least one other pharmaceutically acceptable salt of pseudoephedrine, and the second layer may comprise an antihistamine and/or an expectorant.

The present invention also provides a multi-layered tablet which comprises at least a first layer and a second layer. The first layer comprises codeine and/or dihydrocodeine and/or hydrocodone and/or one or more pharmaceutically acceptable salts thereof and the second layer comprises at least one drug which is selected from decongestants, expectorants, mucus thinning drugs, analgesics, antihistamines and combinations thereof.

In one aspect of the multi-layered tablet, the first layer may be an immediate release layer. In another aspect, the first layer may be a controlled release layer. In yet another aspect, the second layer may be a controlled release layer.

In a still further aspect of the multi-layered tablet of the present invention, the first layer may comprise codeine phosphate and/or dihydrocodeine bitartrate and/or hydrocodone bitartrate.

In another aspect of the multi-layered tablet, the first layer may not contain any active ingredient which is different from codeine, dihydrocodeine, hydrocodone and pharmaceutically acceptable salts thereof.

In another aspect, the multi-layered tablet may comprise at least one, e.g., at least two, active ingredients which are selected from dextromethorphan, phenylepherine, pseudoephedrine, guaifenesin, chlorpheniramine, carbinoxamine, promethazine and pharmaceutically acceptable salts thereof.

In yet another aspect, the at least one drug in the second layer has a plasma half-life which may differ by at least about 1 hour from the plasma half-life of the codeine and/or dihydrocodeine and/or hydrocodone and/or pharmaceutically acceptable salts thereof.

In another aspect, the multi-layered tablet may provide a plasma concentration within the therapeutic range of the at least one drug in the second layer over a period which is coextensive with at least about 80% of the period over which the tablet provides a plasma concentration within the therapeutic range of the codeine and/or dihydrocodeine and/or hydrocodone and/or pharmaceutically acceptable salts thereof.

In yet another aspect, the at least one drug in the second layer may comprise one or more of phenylepherine, pseudoephedrine, chlorpheniramine and pharmaceutically acceptable salts thereof.

In a further aspect of the multi-layered tablet, the layers thereof may be discrete zones which are arranged adjacent to each other; or the second layer may be partially or completely surrounded by the first layer; or the second layer may be coated with the first layer or the first layer may be partially or completely surrounded by the second layer or the first layer may be coated with the second layer.

The present invention further provides a liquid dosage form which comprises (a) codeine and/or dihydrocodeine and/or hydrocodone and/or one or more pharmaceutically acceptable salts thereof, and (b) at least one drug which is selected from decongestants, expectorants, mucus thinning drugs, and antihistamines and combinations thereof. The liquid dosage form provides a plasma concentration within the therapeutic range of (b) over a period which is coextensive with at least about 70% of the period over which the liquid dosage form provides a plasma concentration within the therapeutic range of (a).

In one aspect, the liquid dosage form may comprise a suspension, for example, in the form of a gel.

In another aspect, at least a part of (b) and/or at least a part of (a) may be present as a complex with a complexing agent. By way of non-limiting example, the complexing agent may comprise an ion-exchange resin such as, e.g., sodium polystyrene sulfonate.

In another aspect of the liquid dosage form which comprises a suspension, the suspension may comprise particles of a complex of at least a part of component (b) with an ion-exchange resin, which particles may be provided, at least in part, with a controlled release coating. The controlled release coating may comprise an organic polymer such as, e.g., a polyacrylate.

The present invention also provides a method of concurrently alleviating (including treating) a condition which can be alleviated by administering codeine, dihydrocodeine and/or hydrocodone and at least one other condition which can be alleviated by administering a drug which is a decongestant, expectorant, mucus thinning drug, and/or antihistamine. This method comprises the administration of any of the pharmaceutical dosage forms of the present invention to a subject in need thereof.

In one aspect of the method, the condition which can be alleviated by administering codeine, dihydrocodeine and/or hydrocodone may comprise (excessive) coughing.

In another aspect, the dosage form may be administered not more than about three times per day, e.g., not more than about twice per day.

The present invention further provides a process of making a pharmaceutical dosage form of the present invention, wherein the method comprises preparing a first composition which comprises the first drug (i.e., at least one morphine derivative exhibiting antitussive activity) and a second composition which comprises the at least one second drug, and combining the first and the second compositions to form the dosage form.

In one aspect of the process, the first and second compositions may be combined by using a tablet press.

The present invention also provides a pharmaceutical dosage form which comprises (a) a first drug which comprises codeine and/or dihydrocodeine and/or hydrocodone and/or one or more pharmaceutically acceptable salts thereof and has a first plasma half-life and (b) at least one second drug which is selected from decongestants, expectorants, mucus thinning drugs and antihistamines and has a second plasma half-life which differs from the first plasma half-life by at least about 2 hours, preferably by at least about 3 hours. The dosage form provides a plasma concentration within the therapeutic range of the at least one second drug over a period which is coextensive with at least about 80%, preferably at least about 90%, of the period over which the dosage form provides a plasma concentration within the therapeutic range of the first drug.

In one aspect, the dosage form may comprise a multi-layered tablet. In another aspect, the dosage form may be associated with instructions to administer the dosage form three or fewer times per day, e.g., once or twice per day.

The present invention also provides a pharmaceutical dosage form which comprises (a) at least one first morphine derivative in a first form or layer and (b) at least one second morphine derivative in a second form or layer which is different from or the same as the first form or layer. The dosage form releases the at least one first morphine derivative over a different period and/or at a different rate than the at least one second morphine derivative.

In one aspect of the dosage form, the at least one first morphine derivative and the at least one second morphine derivative may be the same.

In another aspect, the at least one first morphine derivative and the at least one second morphine derivative may independently be selected from codeine, dihydrocodeine, hydrocodone and pharmaceutically acceptable salts thereof.

In yet another aspect, the at least one first morphine derivative and the at least one second morphine derivative may comprise codeine phosphate and/or dihydrocodeine bitartrate and/or hydrocodone bitartrate, preferably codeine phosphate.

In a still further aspect, the first form or layer may be an immediate release form or layer and the second form or layer may be a controlled release form or layer.

In yet another aspect, the dosage form may be a multi-layered tablet which comprises at least one immediate release layer and at least one controlled release layer which independently comprise codeine and/or dihydrocodeine and/or hydrocodone and/or one or more pharmaceutically acceptable salts thereof.

In another aspect, the dosage form may further comprise at least one additional drug which is selected from decongestants, expectorants, mucus thinning drugs, and antihistamines. For example, the at least one additional drug may be present in at least the immediate release layer. Further, the at least one additional drug may be present in at least the controlled release layer.

In yet another aspect, the dosage form may be a liquid (e.g., a suspension) which comprises the at least one first morphine derivative in the free form and the at least one second morphine derivative as a complex with a complexing agent. For example, the complexing agent may comprise an ion-exchange resin.

In yet another aspect, the dosage form may release the at least one first morphine derivative over a different period and at a different rate than the at least one second morphine derivative. In another aspect, it may release the at least one first morphine derivative (at least) over a different period than the at least one second morphine derivative.

In a still further aspect, the dosage form may release the at least one first morphine derivative over a first period and the at least one second morphine derivative over a second period and not more than about 30% of the second period are coextensive with all or a part of the first period. For example, there may be substantially no overlap between the first and second periods.

In a still further aspect, the dosage form may release the at least one first morphine derivative (at least) at a different rate than the at least second morphine derivative.

In another aspect of the dosage form wherein the morphine derivatives are the same, not more than about 30%, preferably not more than about 10% of the period over which a plasma concentration within a therapeutic range is provided by (b) is coextensive with all or a part of a period over which (a) provides a plasma concentration within a therapeutic range, provided that the plasma concentrations provided by (a) and (b) together at any time following ingestion of the dosage form are not higher than the maximum plasma concentration within the therapeutic range of the morphine derivative.

The pharmaceutical dosage form which constitutes one aspect of the present invention comprises a first drug which comprises at least one morphine derivative which exhibits antitussive acitivity (hereafter sometimes referred to as “antitussive morphine derivative”), preferably selected from codeine, dihydrocodeine, hydrocodone (including pharmaceutically acceptable salts thereof) and combinations thereof. A preferred pharmaceutically acceptable salt of codeine is codeine phosphate. In the case of dihydrocodeine or hydrocodone, a preferred salt is the bitartrate. However, other pharmaceutically acceptable salts of these morphine derivatives may be used as well.

The term “pharmaceutically acceptable salts” as used herein and in the appended claims refers to those salts of a particular drug that are not substantially toxic at the dosage administered to achieve the desired effect and do not independently possess significant pharmacological activity. The salts included within the scope of this term are pharmaceutically acceptable acid addition salts of a suitable inorganic or organic acid. Non-limiting examples of suitable inorganic acids are, for example, hydrochloric, hydrobromic, sulfuric and phosphoric acids. Non-limiting examples of suitable organic acids include carboxylic acids, such as acetic, propionic, tannic, glycolic, lactic, pyruvic, malonic, succinic, fumaric, malic, tartaric, citric, cyclamic, ascorbic, maleic, hydroxymaleic, benzoic, phenylacetic, 4-aminobenzoic, 4-hydroxybenzoic, anthranillic, cinnamic, salicylic, 4-aminosalicyclic, 2-phenoxybenzoic, 2-acetoxybenzoic and mandelic acids, as well as sulfonic acids such as, e.g., methanesulfonic, ethanesulfonic, and P-hydroxyethanesulfonic acids.

In addition to the antitussive morphine derivative, the above dosage form contains one or more (e.g., one, two or three) second drugs. Preferred, non-limiting examples of such second drugs are decongestants (such as, e.g., phenylepherine, pseudoephedrine and pharmaceutically acceptable salts thereof), expectorants and mucus thinning drugs (such as, e.g., guaifenesin), and antihistamines (such as, e.g., chlorpheniramine, carbinoxamine, promethazine and pharmaceutically acceptable salts thereof).

The above dosage form provides a plasma concentration within the therapeutic range of the at least one second drug over a period which is coextensive with (overlaps) at least about 70%, more preferred at least about 80%, e.g., at least about 90%, at least about 95%, or about 100%, of the period over which the dosage form provides a plasma concentration within the therapeutic range of the antitussive morphine derivative(s).

The term “therapeutic range” as used herein and in the appended claims refers to the range of drug levels within which most patients will experience a significant therapeutic effect (including alleviation of symptoms) without an undesirable degree of adverse reactions. It is noted that the term “coextensive with” does not exclude, but rather includes, cases where a part of the period over which the plasma concentration of the at least one second drug is within the therapeutic range is outside the period over which the plasma concentration of the antitussive morphine derivative(s) is within the therapeutic range. In other words, even if the corresponding period for the at least one second drug is to overlap, for example, 70% of the corresponding period of the first drug, a certain percentage (preferably not more than about 30%, e.g., not more than about 20%, not more than about 10% or even not more than about 5%) of the total period over which the plasma concentration of the at least one second drug is within the therapeutic range may be outside the period over which the plasma concentration of the antitussive morphine derivative(s) is within the therapeutic range.

The period over which the therapeutic range of a particular drug may be provided in a given case depends, at least in part, on the plasma half-life of the drug and/or active metabolites thereof. The term “plasma half-life” as used herein and in the appended claims refers to the time required for the plasma drug concentration to decline by 50%. The shorter the plasma half-life of a particular drug, the shorter will be the period within the therapeutic range of the drug which is provided by a single administered dose of the drug. In one preferred aspect of the dosage form of the present invention, the plasma half-life of the at least one second drug will be shorter or longer than the plasma half-life of the antitussive morphine derivative(s) by at least about 0.5 hours, e.g., by at least about 1 hour, or at least about 2 hours, but usually not more than about to 10 hours, e.g., not more than about 8 hours, or not more than about 6 hours.

A preferred, although non-limiting, embodiment of the dosage form of the present invention is a tablet, in particular, a bi-layered tablet. Non-limiting examples of other embodiments of the dosage form of the invention are capsules, pills, chewable tablets, extended/sustained/delayed release single layer matrix tablets, suspensions, solutions, syrups, and suppositories.

The bi-layered tablet which forms another aspect of the present invention comprises two layers. The first layer comprises the antitussive morphine derivative(s), as discussed above. The second layer comprises at least one additional drug which is preferably selected from decongestants, expectorants, mucus thinning drugs, and antihistamines. Specific and non-limiting examples of such drugs are given above. The bi-layered tablet provides a plasma concentration within the therapeutic range of the at least one additional drug over a period which is coextensive with at least about 70%, preferably at least about 80%, e.g., at least about 90%, at least about 95%, or about 100% of the period over which the bi-layered tablet provides a plasma concentration within the therapeutic range of the antitussive morphine derivative(s).

In a preferred aspect of the bi-layered tablet, the antitussive morphine derivative(s) is (are) the only active ingredient(s) in the first layer. The second layer will usually contain one, two, three or even more additional drugs. It is to be understood, however, that the first layer may contain further active ingredients, different from the antitussive morphine derivative(s), e.g., one, two or more additional drugs, preferably selected from decongestants, expectorants, mucus thinning drugs, antihistamines and combinations thereof. Conversely, the second layer may also contain one or more antitussive morphine derivatives, e.g., where the second layer provides a release profile of the antitussive morphine derivative(s) that is different from that provided by the first layer. With regard to the present invention in general, one must understand that it does not matter in which form and/or layer a particular active ingredient is present in the dosage form of the present invention which comprises an antitussive morphine derivative, as long as this form and/or layer is capable of providing a therapeutic effect of this active ingredient over a period which substantially overlaps the period over which the dosage form provides a therapeutic effect of the antitussive morphine derivative.

In another preferred aspect of the bi-layered tablet, the first layer is an immediate release layer or a controlled release layer and the second layer is a controlled release layer. The term “controlled release layer” as used herein and in the appended claims refers to any layer that is not an immediate release layer, i.e., does not release all of an active ingredient contained therein within a relatively short time (for example, within less than 1 hour, e.g., less than 0.75 hours, following ingestion of the dosage form). Accordingly, this term is a generic term which encompasses, e.g., sustained (extended) release layers, pulsed release layers, delayed release layers, and the like. Preferably, the controlled release layer releases the one or more active ingredients contained therein continuously or intermittently and, preferably, in approximately equal amounts per time unit, over an extended period of time such as, e.g., at least about 2 hours, at least about 3 hours, at least about 4 hours, or at least about 6 hours, or at least about 8 hours, or at least about 10 hours, or at least about 11 hours. The desirable length of the time period of continuous or intermittent (e.g., pulsed) release depends, inter alia, on the plasma half-life of the drug and/or an active metabolite thereof. Preferably, the or at least one of the controlled release layers of the bi-layered tablet of the present invention contains the antitussive morphine derivative(s).

When two controlled release layers are present in the bi-layered tablet of the present invention, these layers will usually provide different release profiles. By way of non-limiting example, they will release the active ingredient(s) contained therein at different rates, at different times and/or over different time periods. In this regard, it may be desirable for a particular active ingredient to be present in both layers of the bi-layered tablet of the present invention, e.g., in order to extend the period over which the tablet will provide a therapeutic effect of this active ingredient.

The first layer of the bi-layered tablet of the present invention will usually contain at least about 0.1 mg, preferably at least about 5 mg, e.g., at least about 8 mg, at least about 12 mg, at least about 25 mg, or at least about 30 mg of the antitussive morphine derivative(s). Usually, the first layer will not contain more than about 120 mg, preferably not more than about 90 mg, e.g., not more than about 70 mg, not more than about 60 mg, or not more than about 50 mg of the antitussive morphine derivative(s).

The second layer of the bi-layered tablet preferably is a controlled release layer, in particular, a sustained release layer. The controlled release layer may contain, by way of non-limiting example, (i) chlorpheniramine maleate, usually in an amount which is not less than about 0.1 mg, e.g., not less than about 2 mg, or not less than about 4 mg, but not more than about 16 mg, e.g., not more than about 12 mg, or equivalent amounts of any other pharmaceutically acceptable salts of chlorpheniramine; and/or (ii) promethazine hydrochloride, usually in an amount which is not less than about 0.1 mg. e.g., not less that about 6 mg but not more than about 75 mg, or equivalent amounts of any other pharmaceutically acceptable salts of promethazine; and/or (iii) phenylepherine hydrochloride, usually in an amount which is not less than about 1 mg, e.g., not less than about 10 mg, or not less than about 15 mg, but not more than about 90 mg, e.g., not more than about 75 mg, or not not more than about 50 mg, or equivalent amounts of any other pharmaceutically acceptable salts of phenylepherine; and/or (iv) pseudoephedrine hydrochloride, usually in an amount which is not less than about 1 mg, e.g., not less than about 10 mg, not less than about 25 mg, or not less than about 50 mg, but not more than about 240 mg, e.g., not more than about 150 mg, not more than about 100 mg, or not more than about 70 mg, or equivalent amounts of any other pharmaceutically acceptable salts of pseudoephedrine; and/or (v) guaifenesin, usually in an amount which is not less than about 1 mg, e.g., not less than about 10 mg, not less than about 25 mg, or not less than about 50 mg, but not more than about 2400 mg, e.g., not more than about 1500 mg, or equivalent amounts of a pharmaceutically acceptable salt of guaifenesin; and/or (vi) carbinoxamine maleate, usually in an amount which is not less than about 0.1 mg, e.g., not less that about 6 mg, but not more than about 32 mg, e.g., not more than 24 mg, or equivalent amounts of any other pharmaceutically acceptable salts of carbinoxamine; and/or (vii) one or more antitussive morphine derivatives, usually in an amount as indicated above for the first layer, e.g., not less than about 0.1 mg, but not more than about 120 mg.

Another aspect of the present invention is a multi-layered tablet which comprises at least a first layer and a second layer, but may optionally comprise a third, fourth, fifth, etc. layer. The first layer, which may be an immediate release layer or a controlled release layer, but preferably is a controlled release layer (e.g., a sustained release layer), comprises one or more antitussive morphine derivatives (preferably as the only active ingredient(s) contained therein) and the mandatory second layer which may be an immediate release layer or a controlled release layer, but preferably is a controlled release layer may comprise at least one drug which is selected from decongestants, expectorants, mucus thinning drugs, and antihistamines. If more than one additional drug is to be incorporated in the tablet, the first and/or the second layer may contain all of the additional drugs. Alternatively, a separate (third) layer may be provided for the second additional drug, for example, in cases where it would be difficult to design a controlled release layer which provides a desired release rate for both the first and the second additional drug, or the one or more antitussive morphine derivatives and the second additional drug. Of course, a fourth, fifth, etc. layer may be provided for a third or fourth additional drug, and so on. Alternatively and by way of non-limiting example, the second and a third layer may contain the same drug or drugs, but in different (relative) concentrations and/or incorporated in a different controlled release formulation. In another embodiment, more than one layer (e.g., two or three layers) of the multi-layered tablet of the present invention may contain one or more antitussive morphine derivatives, either alone and/or in combination with any of the other therapeutically active ingredients contained in the dosage form. For example, the one or more antitussive morphine derivatives may be contained in an immediate release layer and in one or more controlled release layers which form a part of the multi-layered tablet of the present invention, or the one or more antitussive morphine derivatives may be contained in two or more controlled release layers. Of course, in this case the different layers will usually provide different release profiles (e.g., different release rates, different release periods, different release times, etc.) of the one or more antitussive morphine derivatives.

The multi-layered tablet of the present invention will usually be made up of two or more distinct layers or discrete zones of granulation compressed together with the individual layers lying on top of one another. Layered tablets have the appearance of a sandwich because the edges of each layer or zone are exposed. These layered tablets may be prepared by compressing a granulation onto a previously compressed granulation. The operation may be repeated to produce multi-layered tablets of more than two layers. In a preferred embodiment of the multi-layered tablet of the present invention, the tablet consists of two layers.

It is to be noted that it is not necessary for the two or more individual layers of the multi-layered tablet of the present invention to lie on top of one another. By way of non-limiting example, a second layer (e.g., a sustained release layer) may be partially or completely surrounded by a first layer (e.g., an immediate release layer). For example, the second layer may be coated with the first layer. In the case of three layers, for example, the third layer may be partially or completely coated with the second layer, which in turn may be partially or completely coated with the first layer. Of course, these are but a few examples of the many different ways in which the various layers of the multi-layered tablet of the present invention can be arranged relative to each other. Moreover, it is to be understood that the tablets of the present invention are not limited to such multi-layered tablets. By way of non-limiting example, the tablet may comprise an immediate release matrix which comprises one or more antitussive morphine derivatives, which matrix has dispersed therein particles of one or more sustained release formulations which have any of the other desired drug(s) and/or one or more antitussive morphine derivatives incorporated therein.

Another aspect of the present invention is formed by a liquid (including a semi-solid) dosage form, preferably a suspension, including a gel, which comprises (a) one or more antitussive morphine derivatives and (b) at least one drug which is selected from decongestants, expectorants, mucus thinning drugs, and antihistamines. This liquid dosage form provides a plasma concentration within the therapeutic range of component (b) over a period which is coextensive with at least about 70%, preferably at least 80%, e.g., at least 90%, of the period over which the liquid dosage form provides a plasma concentration within the therapeutic range of component (a). This may be accomplished in various ways. By way of non-limiting example, one component, for example, component (b) may be incorporated into a solid controlled release formulation. For example, particles of component (b) may be provided with a controlled release coating (e.g. a controlled release coating comprising an organic polymer such as, e.g., a polyacrylate). This formulation may then be comminuted, if necessary, in an appropriate manner (e.g., by milling) to form particles of a size which is small enough to be suitable for being suspended in a pharmaceutically acceptable liquid carrier. The other component, e.g., component (a), on the other hand, may be used as such and/or incorporated as an ion-exchange complex, and/or incorporated in a solid immediate release formulation, comminuted and incorporated into the liquid carrier as well. A non-limiting example of a corresponding procedure is described in the Examples below.

Prior to incorporating components (a) and (b) into a pharmaceutically acceptable liquid carrier to form a liquid dosage form (including a gel form) according to the present invention, at least a part of component (a) and/or at least a part of component (b) may be transformed into a complex with a complexing agent. Non-limiting examples of suitable complexing agents comprise ion-exchange resins such as, e.g., (sodium) polystyrene sulfonate.

In one preferred aspect of the liquid (semi-solid) dosage form of the present invention, the dosage form comprises a gel which may comprise particles of an ion-exchange complex of one or more of the active ingredients and a gel-forming agent such as, e.g., a Carbomer (e.g., Carbopol).

In one aspect, the present invention provides a dosage form which comprises at least one antitussive morphine derivative (e.g., the same antitussive morphine derivative or at least two different antitussive morphine derivatives) in at least two different forms and/or layers. This dosage form does not necessarily contain any further active ingredient(s). By way of non-limiting example, an antitussive morphine derivative, e.g., codeine phosphate, may be present in an immediate release layer and in one (or more) controlled release layer(s) of a multi-layered (e.g., bi-layered) tablet, or it may be present in two (or more) controlled release layer(s) of a multi-layered (e.g., bi-layered) tablet where the controlled release layers provide different release profiles of the antitussive morphine derivative. In particular in the case of a liquid dosage form, the dosage form may contain the antitussive morphine derivative both as such (immediate release) and in a controlled release form (e.g., in the form of an ion-exchange complex and/or coated with a sustained/delayed etc. release coating). For example, by providing the at least one antitussive morphine derivative in different forms/layers, the period over which the antitussive morphine derivative exhibits a therapeutic effect may be extended.

The dosage forms of the present invention can be manufactured by processes which are well known to those of skill in the art. For example, for the manufacture of bi-layered tablets, the active ingredients may be dispersed uniformly into a mixture of excipients, for example, by high shear granulation, low shear granulation, fluid bed granulation, or by blending for direct compression Excipients may include diluents, binders, disintegrants, dispersants, lubricants, glidants, stabilizers, surfactants and colorants. Diluents, also termed “fillers”, are typically used to increase the bulk of a tablet so that a practical size is provided for compression. Non-limiting examples of diluents include lactose, cellulose, microcrystalline cellulose, mannitol, dry starch, hydrolyzed starches, powdered sugar, talc, sodium chloride, silicon dioxide, titanium oxide, dicalcium phosphate dihydrate, calcium sulfate, calcium carbonate, alumina and kaolin. Binders impart cohesive qualities to a tablet formulation and are used to ensure that a tablet remains intact after compression. Non-limiting examples of suitable binders include starch (including corn starch and pregelatinized starch), gelatin, sugars (e.g., glucose, dextrose, sucrose, lactose and sorbitol), celluloses, polyethylene glycol, waxes, natural and synthetic gums, e.g., acacia, tragacanth, sodium alginate, and synthetic polymers such as polymethacrylates and polyvinylpyrrolidone. Lubricants facilitate tablet manufacture; non-limiting examples thereof include magnesium stearate, calcium stearate, stearic acid, glyceryl behenate, and polyethylene glycol. Disintegrants facilitate tablet disintegration after administration, and non-limiting examples thereof include starches, alginic acid, crosslinked polymers such as, e.g., crosslinked polyvinylpyrrolidone, croscarmellose sodium, potassium or sodium starch glycolate, clays, celluloses, starches, gums and the like. Non-limiting examples of suitable glidants include silicon dioxide, talc and the like. Stabilizers inhibit or retard drug decomposition reactions, including oxidative reactions. Surfactants may be anionic, cationic, amphoteric or nonionic. If desired, the tablets may also contain minor amounts of nontoxic auxiliary substances such as pH buffering agents, preservatives, e.g., antioxidants, wetting or emulsifying agents, solubilizing agents, coating agents, flavoring agents, and the like.

Extended/sustained release formulations may be made by choosing the right combination of excipients that slow the release of the active ingredients by coating or temporarily bonding or decreasing the solubility of the active ingredients. Examples of these excipients include cellulose ethers such as hydroxypropylmethylcellulose (e.g., Methocel K4M), polyvinylacetate-based excipients such as, e.g., Kollidon SR, and polymers and copolymers based on methacrylates and methacrylic acid such as, e.g., Eudragit NE 30D.

There are several commercially available tablet presses capable of making bi-layered tablets. For example, Manesty RotaPress Diamond, a 45 station D tooling press, is capable of making bi-layered tablets described in this application. Non-limiting examples of presses for the manufacture of bi-layered tablets include Fette America Model No. PT 3090; Maneklal Global Exports (Mumbai, India) Models JD and DH series; Niro Pharma Systems, Model R292F; and Korsch AG Models XL 800 and XL 400.

### DETAILED DESCRIPTION OF THE PRESENT INVENTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description making apparent to those skilled in the art how the several forms of the present invention may be embodied in practice.

The following Examples illustrate the use of codeine phosphate as an antitussive morphine derivative. It is to be understood that the codeine phosphate in these Examples may be replaced by equivalent amounts of any other antitussive morphine derivative (e.g., those set forth above) or combinations thereof.

### EXAMPLE 1

### Bi-Layered Tablet (Wet Granulation)

A bi-layered tablet in accordance with the present invention which comprises guaifenesin in a first sustained release layer and codeine phosphate and pseudoephedrine hydrochloride in a second sustained release layer is illustrated as follows:

| | | |
|---|---|---|
| | Weight/tablet | Weight/1 kg |
| Ingredients | (mgs) | batch (gms) |

Layer 1 (Sustained release)

| | | |
|---|---|---|
| Guaifenesin | 600.0 | 510.6 |
| Methocel K15M | 100.0 | 85.1 |
| Silicified Microcrystalline Cellulose | 50 | 42.6 |
| Eudragit NE | 42 | 35.7 |
| Magnesium Stearate | 8.0 | 6.8 |

Layer 2 (Sustained release)

| | | |
|---|---|---|
| Codeine Phosphate | 30.0 | 25.5 |
| Pseudoephedrine HCl | 120.0 | 102.1 |
| Microcrystalline Cellulose (PH 102) | 45.0 | 38.3 |
| Eudragit NE | 15.0 | 12.8 |
| Methocel K4M Premium | 140.0 | 119.1 |
| Stearic Acid | 20.0 | 17.0 |
| Magnesium Stearate | 5.0 | 4.3 |
| Total | 1175.0 | 1000.0 |

Procedure:
(a) Sustained release layer #1: Mix the guaifenesin, Methocel®K15M and silicified microcrystalline cellulose in a high shear mixer/granulator for 10 minutes. Granulate the above blend using a Eudragit® NE (30%). Dry the granulation until the LOD (weight loss on drying) is less than 2.0%. Screen granules through a USP sieve size # 14. Add the granules and the prescreened magnesium stearate in a V shaped blender and mix for 3 minutes.

(b) Sustained release layer #2: Screen all ingredients through a USP sieve size # 30. Mix the codeine phosphate, pseudoephedrine HCI, microcrystalline cellulose PH 102, and stearic acid in a high shear mixer/granulator for 10 minutes. Granulate the above blend using a Eudragit® NE (30%). Add the Methocel®K4M to the granulator and post mix for 5 minutes. Dry the granulation until the LOD is less than 2.0%. Screen granules through a USP sieve size # 14. Add the granules and the prescreened magnesium stearate in a V shaped blender and mix for 3 minutes.

Manufacture bi-layered tablets using a rotary bi-layer tablet press where in each tablet layer #1 is 800 mgs and layer #2 is 375 mgs. Capsules may be manufactured by filling the same proportions into capsules.

### EXAMPLE 2

### Bi-Layered Tablet (Wet Granulation)

A bi-layered tablet in accordance with the present invention which comprises promethazine hydrochloride in an immediate release layer and codeine phosphate and pseudoephedrine hydrochloride in a sustained release layer is illustrated as follows:

| | | |
|---|---|---|
| | Weight/tablet | Weight/1 kg |
| Ingredients | (mgs) | batch (gms) |

Layer 1 (Immediate release)

| | | |
|---|---|---|
| Promethazine HCl | 25.0 | 37.0 |
| Silicified Microcrystalline Cellulose | 111.0 | 164.3 |
| Povidone | 3.0 | 4.4 |
| Croscarmellose Sodium | 10.0 | 14.8 |
| Magnesium Stearate | 1.0 | 1.5 |

Layer 2 (Sustained release)

| | | |
|---|---|---|
| Codeine Phosphate | 30.0 | 44.4 |
| Pseudoephedrine HCl | 120.0 | 177.6 |
| Microcrystalline Cellulose (PH 102) | 30.0 | 44.4 |
| Dicalcium Phosphate | 100.0 | 148.0 |
| Povidone | 15.0 | 22.2 |
| Methocel K4M Premium | 205.0 | 303.4 |
| Stearic Acid | 20.0 | 29.6 |
| Magnesium Stearate | 5.0 | 7.4 |
| Total | 675.0 | 1000.0 |

Procedure:

(a) Immediate release layer #1: Mix the promethazine HCl, silicified microcrystalline cellulose and croscarmellose sodium, in a high shear mixer/granulator for 10 minutes. Granulate the above blend using a 30% povidone solution (3.0 gms povidone in 10.0 gms purified water). Dry the granulation until the LOD is less than 2.0%. Screen granules through a USP sieve size # 14. Add the granules and the prescreened magnesium stearate in a V shaped blender and mix for 3 minutes.

(b) Sustained release layer #2: Mix the codeine phosphate, pseudoephedrine HCl, microcrystalline cellulose PH 102, dicalcium phosphate, Methocel K4M Premium and stearic acid in a high shear mixer/granulator for 10 minutes. Granulate the above blend using a 30% povidone solution (15.0 gms povidone in 50.0 gms purified water). Dry the granulation until the LOD is less than 2.0%. Screen granules through a USP sieve size # 14. Add the granules and the prescreened magnesium stearate in a V shaped blender and mix for 3 minutes.

Manufacture bi-layered tablets using a rotary bi-layer tablet press where in each tablet layer #1 is 150 mgs and layer #2 is 525 mgs. Capsules may be manufactured by filling the same proportions into capsules.

### EXAMPLE 3

### Bi-Layered Tablet (Wet Granulation)

A bi-layered tablet in accordance with the present invention which comprises phenylepherine hydrochloride and carbinoxamine maleate in a first sustained release layer and codeine phosphate in a second sustained release layer is illustrated as follows:

| | | |
|---|---|---|
| | Weight/tablet | Weight/1 kg |
| Ingredients | (mgs) | batch (gms) |

Layer 1 (Sustained release)

| | | |
|---|---|---|
| Phenylepherine HCl | 75.0 | 185.2 |
| Carbinoxamine Maleate | 8.0 | 19.8 |
| Methocel K4M | 59.0 | 145.7 |
| Silicified Microcrystalline Cellulose | 30.0 | 74.1 |
| Eudragit NE | 15.0 | 37.0 |
| Magnesium Stearate | 3.0 | 7.4 |

Layer 2 (Sustained release)

| | | |
|---|---|---|
| Codeine Phosphate | 30.0 | 74.1 |
| Microcrystalline Cellulose (PH 102) | 45.0 | 111.1 |
| Eudragit NE | 15.0 | 37.0 |
| Methocel K4M Premium | 100.0 | 246.9 |
| Stearic Acid | 20.0 | 49.4 |
| Magnesium Stearate | 5.0 | 12.3 |
| Total | 405.0 | 1000.0 |

Procedure:

(a) Sustained release layer #1: Mix the phenylepherine HCl, carbinoxamine maleate, Methocel®K4M and silicified microcrystalline cellulose in a high shear mixer/granulator for 10 minutes. Granulate the above blend using a Eudragit® NE (30%). Dry the granulation until the LOD is less than 2.0%. Screen granules through a USP sieve size # 14. Add the granules and the prescreened magnesium stearate in a V shaped blender and mix for 3 minutes.

(b) Sustained release layer #2: Screen all ingredients through a USP sieve size # 30. Mix the codeine phosphate, microcrystalline cellulose PH 102, and stearic acid in a high shear mixer/granulator for 10 minutes. Granulate the above blend using a Eudragit® NE (30%). Add the Methocel®K4M to the granulator and post mix for 5 minutes. Dry the granulation until the LOD is less than 2.0%. Screen granules through a USP sieve size # 14. Add the granules and the prescreened magnesium stearate in a V shaped blender and mix for 3 minutes.

Manufacture bi-layered tablets using a rotary bi-layer tablet press where in each tablet layer #1 is 190 mgs and layer #2 is 215 mgs. Capsules may be manufactured by filling the same proportions into capsules.

### EXAMPLE 4

### Bi-Layered Tablet (Wet Granulation)

A bi-layered tablet in accordance with the present invention which comprises pseudoephedrine hydrochloride and chlorpheniramine maleate in a first sustained release layer and codeine phosphate in a second sustained release layer is illustrated as follows:

| | | |
|---|---|---|
| | Weight/tablet | Weight/1 kg |
| Ingredients | (mgs) | batch (gms) |

Layer 1 (Sustained release)

| | | |
|---|---|---|
| Pseudoephedrine HCl | 120.0 | 253.2 |
| Chlorpheniramine Maleate | 12.0 | 25.3 |
| Methocel K4M | 70.0 | 147.7 |
| Silicified Microcrystalline Cellulose | 35.0 | 73.9 |
| Eudragit NE | 20.0 | 42.2 |
| Magnesium Stearate | 3.0 | 6.3 |

Layer 2 (Sustained release)

| | | |
|---|---|---|
| Codeine Phosphate | 30.0 | 63.3 |
| Microcrystalline Cellulose (PH 102) | 45.0 | 95.0 |
| Eudragit NE | 15.0 | 31.7 |
| Methocel K4M Premium | 100.0 | 211.0 |
| Stearic Acid | 20.0 | 42.2 |
| Magnesium Stearate | 5.0 | 10.6 |
| Total | 475.0 | 1000.0 |

Procedure:

(a) Sustained release layer #1: Mix the pseudoephedrine HCl, chlorpheniramine maleate, Methocel®K4M and silicified microcrystalline cellulose in a high shear mixer/granulator for 10 minutes. Granulate the above blend using a Eudragit® NE (30%). Dry the granulation until the LOD is less than 2.0%. Screen granules through a USP sieve size # 14. Add the granules and the prescreened magnesium stearate in a V shaped blender and mix for 3 minutes.

(b) Sustained release layer #2: Screen all ingredients through a USP sieve size # 30. Mix the codeine phosphate, microcrystalline cellulose PH 102, and stearic acid in a high shear mixer/granulator for 10 minutes. Granulate the above blend using a Eudragit® NE (30%). Add the Methocel®K4M to the granulator and post mix for 5 minutes. Dry the granulation until the LOD is less than 2.0%. Screen granules through a USP sieve size # 14. Add the granules and the prescreened magnesium stearate in a V shaped blender and mix for 3 minutes.

Manufacture bi-layered tablets using a rotary bi-layer tablet press where in each tablet layer #1 is 260 mgs and layer #2 is 215 mgs. Capsules may be manufactured by filling the same proportions into capsules.

### EXAMPLE 5

### Bi-Layered Tablet (Wet Granulation)

A bi-layered tablet in accordance with the present invention which comprises carbinoxamine maleate in a first sustained release layer and codeine phosphate in a second sustained release layer is illustrated as follows:

| | | |
|---|---|---|
| | Weight/tablet | Weight/1 kg |
| Ingredients | (mgs) | batch (gms) |

Layer 1 (Sustained release)

| | | |
|---|---|---|
| Carbinoxamine Maleate | 8.0 | 19.3 |
| Lactose Monohydrate | 61.0 | 147.0 |
| Methocel K4M | 70.0 | 168.7 |
| Silicified Microcrystalline Cellulose | 39.0 | 94.0 |
| Eudragit NE | 20.0 | 48.2 |
| Magnesium Stearate | 2.0 | 4.82 |

Layer 2 (Sustained release)

| | | |
|---|---|---|
| Codeine Phosphate | 30.0 | 72.3 |
| Microcrystalline Cellulose (PH 102) | 45.0 | 108.5 |
| Eudragit NE | 15.0 | 36.2 |
| Methocel K4M Premium | 100.0 | 241.0 |
| Stearic Acid | 20.0 | 48.2 |
| Magnesium Stearate | 5.0 | 12.1 |
| Total | 415.0 | 1000.0 |

Procedure:

(a) Sustained release layer #1: Mix the carbinoxamine maleate, Methocel®K4M, lactose monohydrate and silicified microcrystalline cellulose in a high shear mixer/granulator for 10 minutes. Granulate the above blend using a Eudragit® NE (30%). Dry the granulation until the LOD is less than 2.0%. Screen granules through a USP sieve size # 14. Add the granules and the prescreened magnesium stearate in a V shaped blender and mix for 3 minutes.

(b) Sustained release layer #2: Screen all ingredients through a USP sieve size # 30. Mix the codeine phosphate, microcrystalline cellulose PH 102, and stearic acid in a high shear mixer/granulator for 10 minutes. Granulate the above blend using a Eudragit® NE (30%). Add the Methocel®K4M to the granulator and post mix for 5 minutes. Dry the granulation until the LOD is less than 2.0%. Screen granules through a USP sieve size # 14. Add the granules and the prescreened magnesium stearate in a V shaped blender and mix for 3 minutes.

Manufacture bi-layered tablets using a rotary bi-layer tablet press where in each tablet layer #1 is 200 mgs and layer #2 is 215 mgs. Capsules may be manufactured by filling the same proportions into capsules.

### EXAMPLE 6

### Bi-Layered Tablet (Direct Compression)

A bi-layered tablet in accordance with the present invention which comprises promethazine hydrochloride (longer half-life drug) in an immediate release layer and codeine phosphate (shorter half-life drug) in a sustained release layer is illustrated as follows:

| | | |
|---|---|---|
| | Weight/tablet | Weight/1 kg batch |
| Ingredients | (mg) | (in grams) |

Layer 1 (Immediate release)

| | | |
|---|---|---|
| Promethazine HCl | 25 | 45.5 |
| Silicified Microcrystalline | 114.0 | 207.5 |
| Cellulose | | |
| Sodium Starch Glycolate | 10.0 | 18.2 |
| Magnesium Stearate | 1.0 | 1.8 |

Layer 2 (Sustained release)

| | | |
|---|---|---|
| Codeine Phosphate | 60.0 | 109.2 |
| Lactose Monohydrate | 50.0 | 91.0 |
| Dicalcium Phosphate | 50.0 | 91.0 |
| Kollidon SR | 220.0 | 400.4 |
| Stearic acid | 15.0 | 27.3 |
| Magnesium Stearate | 5.0 | 9.1 |
| Total | 550.0 | 1000.0 |

Procedure:

(a) Immediate release layer #1: Screen all ingredients through a USP sieve size # 30. Blend the promethazine hydrochloride, microcrystalline cellulose and sodium starch glycolate for 20 minutes. Add magnesium stearate to the above blend and mix for an additional time of three minutes.

(b) Sustained release layer #2: Blend the codeine phosphate, lactose monohydrate, dicalcium phosphate and Kollidon® SR for 20 minutes. Add stearic acid and magnesium stearate to the above blend and mix for an additional time of three minutes.

Manufacture bi-layered tablets using a rotary bi-layer tablet press where in each tablet the immediate release layer #1 is 150 mgs and the sustained release layer #2 is 400 mgs. Capsules may be manufactured by filling the same proportions into capsules.

### EXAMPLE 7

### Bi-Layered Tablet (Wet Granulation)

A bi-layered tablet in accordance with the present invention which comprises pseudoephedrine tannate and chlorpheniramine tannate in an immediate release layer and codeine phosphate in a sustained release layer is illustrated as follows:

| | | |
|---|---|---|
| | Weight/tablet | Weight/1 kg |
| Ingredients | (mgs) | batch (gms) |

Layer 1 (Immediate release)

| | | |
|---|---|---|
| Pseudoephedrine Tannate | 60.0 | 85.7 |
| Chlorpheniramine Tannate | 8.0 | 11.4 |
| Silicified Microcrystalline Cellulose | 108.0 | 154.3 |
| Povidone | 3.0 | 4.3 |
| Croscarmellose Sodium | 10.0 | 14.3 |
| Magnesium Stearate | 1.0 | 1.4 |

Layer 2 (Sustained release)

| | | |
|---|---|---|
| Codeine Phosphate | 30.0 | 42.9 |
| Microcrystalline Cellulose (PH 102) | 30.0 | 42.9 |
| Lactose Monohydrate | 100.0 | 142.9 |
| Dicalcium Phosphate | 100.0 | 142.9 |
| Povidone | 15.0 | 21.4 |
| Methocel K4M Premium | 210.0 | 300.0 |
| Stearic Acid | 20.0 | 28.6 |
| Magnesium Stearate | 5.0 | 7.1 |
| Total | 700.0 | 1000.0 |

Procedure:

(a) Immediate release layer #1: Mix the pseudoephedrine tannate, chlorpheniramine tannate, silicified microcrystalline cellulose and croscarmellose sodium, in a high shear mixer/granulator for 10 minutes. Granulate the above blend using a 30% povidone solution (3.0 gms povidone in 10.0 gms purified water). Dry the granulation until the LOD is less than 2.0%. Screen granules through a USP sieve size # 14. Add the granules and the prescreened magnesium stearate in a V shaped blender and mix for 3 minutes.

(b) Sustained release layer #2: Mix the codeine phosphate, microcrystalline cellulose PH 102, lactose monohydrate, dicalcium phosphate, Methocel K4M Premium and stearic acid in a high shear mixer/granulator for 10 minutes. Granulate the above blend using a 30% povidone solution (15.0 gms povidone in 50.0 gms purified water). Dry the granulation until the LOD is less than 2.0%. Screen granules through a USP sieve size # 14. Add the granules and the prescreened magnesium stearate in a V shaped blender and mix for 3 minutes.

Manufacture bi-layered tablets using a rotary bi-layer tablet press where in each tablet layer #1 is 190 mgs and layer #2 is 510 mgs. Capsules may be manufactured by filling the same proportions into capsules.

### EXAMPLE 8

### Bi-Layered Tablet (Wet Granulation)

A bi-layered tablet in accordance with the present invention which comprises promethazine hydrochloride in an immediate release layer and codeine phosphate and phenylepherine hydrochloride in a sustained release layer is illustrated as follows:

| | | |
|---|---|---|
| | Weight/tablet | Weight/1 kg |
| Ingredients | (mgs) | batch (gms) |

Layer 1 (Immediate release)

| | | |
|---|---|---|
| Promethazine HCl | 25 | 55.5 |
| Silicified Microcrystalline Cellulose | 86.0 | 190.0 |
| Povidone | 3.0 | 6.7 |
| Croscarmellose Sodium | 10.0 | 22.2 |
| Magnesium Stearate | 1.0 | 2.2 |

Layer 2 (Sustained release)

| | | |
|---|---|---|
| Codeine Phosphate | 30.0 | 66.6 |
| Phenylepherine HCl | 75.0 | 166.5 |
| Microcrystalline Cellulose (PH 102) | 30.0 | 66.6 |
| Dicalcium Phosphate | 30.0 | 66.6 |
| Povidone | 15.0 | 33.3 |
| Methocel K4M Premium | 120.0 | 266.4 |
| Stearic Acid | 20.0 | 44.4 |
| Magnesium Stearate | 5.0 | 11.1 |
| Total | 450.0 | 1000.0 |

Procedure:

(a) Immediate release layer #1: Mix the promethazine hydrochloride, silicified microcrystalline cellulose and croscarmellose sodium, in a high shear mixer/granulator for 10 minutes. Granulate the above blend using a 30% povidone solution (3.0 gms povidone in 10.0 gms purified water). Dry the granulation until the LOD is less than 2.0%. Screen granules through a USP sieve size # 14. Add the granules and the prescreened magnesium stearate in a V shaped blender and mix for 3 minutes.

(b) Sustained release layer #2: Mix the codeine phosphate, phenylepherine HCl, microcrystalline cellulose PH 102, dicalcium phosphate, Methocel K4M Premium and stearic acid in a high shear mixer/granulator for 10 minutes. Granulate the above blend using a 30% povidone solution (15.0 gms povidone in 50.0 gms purified water). Dry the granulation until the LOD is less than 2.0%. Screen granules through a USP sieve size # 14. Add the granules and the prescreened magnesium stearate in a V shaped blender and mix for 3 minutes.

Manufacture bi-layered tablets using a rotary bi-layer tablet press where in each tablet layer #1 is 125 mgs and layer #2 is 325 mgs. Capsules may be manufactured by filling the same proportions into capsules.

### EXAMPLE 9

### Bi-Layered Tablet (Direct Compression)

A bi-layered tablet in accordance with the present invention which comprises guaifenesin in a first sustained release layer and codeine phosphate in a second sustained release layer is illustrated as follows:

| | | |
|---|---|---|
| | Weight/tablet | Weight/1 kg batch |
| Ingredients | (mg) | (in grams) |

Layer 1 (Sustained release)

| | | |
|---|---|---|
| Guaifenesin | 600.0 | 499.8 |
| Methocel K15M | 200.0 | 166.6 |
| Silicified Microcrystalline | 72 | 60.0 |
| Cellulose | | |
| Magnesium Stearate | 8.0 | 6.7 |

Layer 2 (Sustained release)

| | | |
|---|---|---|
| Codeine Phosphate | 60.0 | 50.0 |
| Lactose Monohydrate | 35.0 | 29.2 |
| Dicalcium Phosphate | 35.0 | 29.2 |
| Kollidon SR | 170.0 | 141.6 |
| Stearic acid | 15.0 | 12.5 |
| Magnesium Stearate | 5.0 | 4.2 |
| Total | 1200.0 | 1000.0 |

Procedure:

(a) Sustained release layer #1: Screen all ingredients through a USP sieve size # 30. Blend the guaifenesin, Methocel® K15M and silicified microcrystalline cellulose for 25 minutes. Add magnesium stearate to the above blend and mix for an additional time of three minutes.

(b) Sustained release layer #2: Blend the codeine phosphate, lactose monohydrate, dicalcium phosphate and Kollidon® SR for 20 minutes. Add stearic acid and magnesium stearate to the above blend and mix for an additional time of three minutes.

Manufacture bi-layered tablets using a rotary bi-layer tablet press where in each tablet layer #1 is 880 mgs and layer #2 is 320 mgs. Capsules may be manufactured by filling the same proportions into capsules.

### EXAMPLE 10

### Bi-Layered Tablet (Wet Granulation)

A bi-layered tablet in accordance with the present invention which comprises guaifenesin in a first sustained release layer and codeine phosphate and phenylepherine hydrochloride in a second sustained release layer is illustrated as follows:

| | | |
|---|---|---|
| | Weight/tablet | Weight/1 kg |
| Ingredients | (mgs) | batch (gms) |

Layer 1 (Sustained release)

| | | |
|---|---|---|
| Guaifenesin | 600.0 | 558.0 |
| Methocel K15M | 100.0 | 93.0 |
| Silicified Microcrystalline Cellulose | 50 | 46.5 |
| Eudragit NE | 42 | 39.1 |
| Magnesium Stearate | 8.0 | 7.4 |

Layer 2 (Sustained release)

| | | |
|---|---|---|
| Codeine Phosphate | 30.0 | 27.9 |
| Phenylepherine HCl | 60.0 | 55.8 |
| Microcrystalline Cellulose (PH 102) | 45.0 | 41.9 |
| Eudragit NE | 15.0 | 14.0 |
| Methocel K4M Premium | 100.0 | 93.0 |
| Stearic Acid | 20.0 | 18.6 |
| Magnesium Stearate | 5.0 | 4.7 |
| Total | 1075.0 | 1000.0 |

Procedure:

(a) Sustained release layer #1: Mix the guaifenesin, Methocel®K15M and silicified microcrystalline cellulose in a high shear mixer/granulator for 10 minutes. Granulate the above blend using a Eudragit® NE (30%). Dry the granulation until the LOD is less than 2.0%. Screen granules through a USP sieve size # 14. Add the granules and the prescreened magnesium stearate in a V shaped blender and mix for 3 minutes.

(b) Sustained release layer #2: Screen all ingredients through a USP sieve size # 30. Mix the codeine phosphate, phenylepherine HCl, microcrystalline cellulose PH 102, dicalcium phosphate and stearic acid in a high shear mixer/granulator for 10 minutes. Granulate the above blend using a Eudragit® NE (30%). Add the Methocel®K4M to the granulator and post mix for 5 minutes. Dry the granulation until the LOD is less than 2.0%. Screen granules through a USP sieve size # 14. Add the granules and the prescreened magnesium stearate in a V shaped blender and mix for 3 minutes. controlled release layer(s) of a multi-layered (e.g., bi-layered) tablet.

Manufacture bi-layered tablets using a rotary bi-layer tablet press where in each tablet layer #1 is 800 mgs and layer #2 is 275 mgs. Capsules may be manufactured by filling the same proportions into capsules.

### EXAMPLE 11

### Bi-Layered Tablet (Wet Granulation)

A bi-layered tablet in accordance with the present invention which comprises guaifenesin in a first sustained release layer and codeine phosphate and phenylepherine hydrochloride in a second sustained release layer is illustrated as follows:

| | | |
|---|---|---|
| | Weight/tablet | Weight/1 kg |
| Ingredients | (mgs) | batch (gms) |

Layer 1 (Sustained release)

| | | |
|---|---|---|
| Guaifenesin | 1000.0 | 635.0 |
| Methocel K15M | 200.0 | 127.0 |
| Silicified Microcrystalline Cellulose | 40.0 | 25.4 |
| Eudragit NE | 50.0 | 31.8 |
| Magnesium Stearate | 10.0 | 6.4 |

Layer 2 (Sustained release)

| | | |
|---|---|---|
| Codeine Phosphate | 30.0 | 19.1 |
| Phenylepherine HCl | 60.0 | 38.1 |
| Microcrystalline Cellulose (PH 102) | 45.0 | 28.6 |
| Eudragit NE | 15.0 | 9.5 |
| Methocel K4M Premium | 100.0 | 63.5 |
| Stearic Acid | 20.0 | 12.7 |
| Magnesium Stearate | 5.0 | 3.2 |
| Total | 1575.0 | 1000.0 |

Procedure:

(a) Sustained release layer #1: Mix the guaifenesin, Methocel®K15M and silicified microcrystalline cellulose in a high shear mixer/granulator for 10 minutes. Granulate the above blend using a Eudragit® NE (30%). Dry the granulation until the LOD is less than 2.0%. Screen granules through a USP sieve size # 14. Add the granules and the prescreened magnesium stearate in a V shaped blender and mix for 3 minutes.

(b) Sustained release layer #2: Screen all ingredients through a USP sieve size # 30. Mix the codeine phosphate, phenylepherine HCl, microcrystalline cellulose PH 102, dicalcium phosphate and stearic acid in a high shear mixer/granulator for 10 minutes. Granulate the above blend using a Eudragit® NE (30%). Add the Methocel®K4M to the granulator and post mix for 5 minutes. Dry the granulation until the LOD is less than 2.0%. Screen granules through a USP sieve size # 14. Add the granules and the prescreened magnesium stearate in a V shaped blender and mix for 3 minutes.
controlled release layer(s) of a multi-layered (e.g., bi-layered) tablet Manufacture bi-layered tablets using a rotary bi-layer tablet press where in each tablet layer #1 is 1300 mgs and layer #2 is 275 mgs. Capsules may be manufactured by filling the same proportions into capsules.

### EXAMPLE 12

### Bi-Layered Tablet (Direct Compression)

A bi-layered tablet in accordance with the present invention which comprises codeine phosphate in a first sustained release layer and phenylepherine hydrochloride and chlorpheniramine maleate in a second sustained release layer is illustrated as follows:

| | | |
|---|---|---|
| | Weight/tablet | Weight/1 kg batch |
| Ingredients | (mg) | (in grams) |

Layer 1 (Sustained release)

| | | |
|---|---|---|
| Codeine Phosphate | 30 | 54.5 |
| Methocel K4M | 50 | 90.9 |
| Silicified Microcrystalline | 100.0 | 181.8 |
| Cellulose | | |
| Sodium Starch Glycolate | 10.0 | 18.2 |
| Magnesium Stearate | 1.0 | 1.8 |

Layer 2 (Sustained release)

| | | |
|---|---|---|
| Phenylepherine HCl | 60 | 109 |
| Chlorpheniramine Maleate | 8.0 | 14.5 |
| Lactose Monohydrate | 50.0 | 90.9 |
| Dicalcium Phosphate | 50.0 | 90.9 |
| Methocel K4M | 181.0 | 329.1 |
| Stearic acid | 15.0 | 27.3 |
| Magnesium Stearate | 5.0 | 9.1 |
| Total | 550.0 | 1000.0 |

Procedure:

(a) Sustained release Layer #1: Screen all ingredients through a USP sieve size # 30. Preblend a portion of the Kollidon SR (145 gms) and all the codeine phosphate for 15 minutes. Add lactose monohydrate (90.9 gms) and dicalcium phosphate (90.9 gms) to the above preblend and mix for an additional 20 minutes. Add stearic acid (27.3 gms) and magnesium stearate (9.1 gms) to the above blend and mix for three minutes.

(b) Sustained release layer #2: Screen all ingredients through a USP sieve size # 30. Preblend a portion of the Kollidon SR (145 gms) and all the chlorpheniramine maleate (14.5 gms) for 15 minutes. Add the remaining Kollidon SR (313.2 gms), phenylepherine hydrochloride (36.4 gms), lactose monohydrate (90.9 gms) and dicalcium phosphate (90.9 gms) to the above preblend and mix for an additional 20 minutes. Add stearic acid (27.3 gms) and magnesium stearate (9.1 gms) to the above blend and mix for three minutes.

Manufacture bi-layered tablets using a rotary bi-layer tablet press where in each tablet the immediate release layer is 150 mgs and the sustained release layer is 400 mgs.

### EXAMPLE 13

### Bi-Layered Tablet (Direct Compression)

By using the process described in Example 12, a bi-layered tablet which contains codeine phosphate in an immediate release layer and codeine phosphate, phenylepherine hydrochloride and chlorpheniramine maleate in a sustained release layer may be manufactured by using direct compression:

| | |
|---|---|
| Ingredients | Weight/tablet (mgs) |

Layer 1 (Immediate Release)

| | |
|---|---|
| Codeine Phosphate | 10 |
| Silicified Microcrystalline Cellulose | 133.5 |
| Sodium Starch Glycolate | 15 |
| Magnesium Stearate | 1.5 |

Layer 2 (Sustained Release)

| | |
|---|---|
| Codeine Phosphate | 40 |
| Phenylepherine HCl | 50 |
| Chlorpheniramine Maleate | 8 |
| Lactose Monohydrate | 50 |
| Dicalcium Phosphate | 50 |
| Kollidon SR | 252 |
| Stearic Acid | 15 |
| Magnesium Stearate | 5 |
| Total | 620 |

### EXAMPLE 14

### Bi-Layered Tablet (Wet Granulation)

A bi-layered tablet in accordance with the present invention which comprises codeine phosphate in an immediate release layer and codeine phosphate, pseudoephedrine hydrochloride and chlorpheniramine maleate in a sustained release layer is illustrated as follows:

| | | |
|---|---|---|
| | Weight/tablet | Weight/1 kg |
| Ingredients | (mgs) | batch (gms) |

Layer 1 (Immediate release)

| | | |
|---|---|---|
| Codeine Phosphate | 10.0 | 11.9 |
| Silicified Microcrystalline Cellulose | 111.0 | 158.6 |
| Povidone | 3.0 | 4.3 |
| Croscarmellose Sodium | 10.0 | 14.3 |
| Magnesium Stearate | 1.0 | 1.4 |

Layer 2 (Sustained release)

| | | |
|---|---|---|
| Codeine Phosphate | 30 | 35.7 |
| Pseudoephedrine HCl | 60.0 | 85.7 |
| Chlorpheniramine Maleate | 8.0 | 11.4 |
| Microcrystalline Cellulose (PH 102) | 30.0 | 42.9 |
| Lactose Monohydrate | 100.0 | 142.9 |
| Dicalcium Phosphate | 100.0 | 142.9 |
| Povidone | 15.0 | 21.4 |
| Methocel K4M Premium | 212.0 | 302.9 |
| Stearic Acid | 20.0 | 28.6 |
| Magnesium Stearate | 5.0 | 7.1 |
| Total | 700.0 | 1012.0 |

Procedure:

(a) Immediate release layer #1: Screen all ingredients through a USP sieve size # 30. Blend the codeine phosphate (11.9 grams), silicified microcrystalline cellulose (158.6 grams), and croscarmellose sodium in a high shear mixer/granulator for 10 minutes. Granulate the above blend using a 30% povidone solution (4.3 gms povidone in 14.3 gms purified water). Dry the granulation until the LOD is less than 2.0%. Screen granules through a USP sieve size # 14. Add granules and the prescreened magnesium stearate (1.4 gms) to the above blend and mix for 3 minutes.

(b) Sustained release layer #2: Screen all ingredients through a USP sieve size # 30. Blend the pseudoephedrine hydrochloride (87.5 gms), chlorpheniramine maleate (11.4 gms), codeine phosphate (37.5 gms), microcrystalline cellulose PH 102 (42.9 gms), lactose monohydrate (142.9 gms), dicalcium phosphate (142.9 gms), Methocel K4M Premium (302.9 gms) and stearic acid (28.6 gms) in a high shear mixer/granulator for 10 minutes. Granulate the above blend using a 30% povidone solution (21.4 gms povidone in 71.3 gms purified water). Dry the granulation until the LOD is less than 2.0%. Screen granules through a USP sieve size # 14. Add granules and the prescreened magnesium stearate (7.1 gms) to the above blend and mix for 3 minutes.

Manufacture bi-layered tablets using a rotary bi-layer tablet press where in each tablet the immediate release layer is 150 mgs and the sustained release layer is 550 mgs.

### EXAMPLE 15

### Bi-Layered Tablet (Wet Granulation)

By using the process described in Example 14, a bi-layered tablet containing codeine phosphate in an immediate release layer and pseudoephedrine hydrochloride and chlorpheniramine maleate in a sustained release layer may be manufactured by using wet granulation:

| | |
|---|---|
| Ingredients | Weight/tablet (mgs) |

Layer 1 (Immediate Release)

| | |
|---|---|
| Codeine Phosphate | 30 |
| Silicified Microcrystalline cellulose | 129.5 |
| Povidone | 4 |
| Croscarmellose sodium | 15 |
| Magnesium Stearate | 1.5 |

Layer 2 (Sustained Release)

| | |
|---|---|
| Pseudoephedrine HCl | 60 |
| Chlorpheniramine Maleate | 8 |
| Microcrystalline Cellulose 102 | 30 |
| Lactose Monohydrate | 100 |
| Dicalcium Phosphate | 100 |
| Povidone | 15 |
| Hydroxypropylmethylcellulose | 212 |
| Stearic Acid | 20 |
| Magnesium Stearate | 5 |
| Total | 750 |

The above examples illustrate how to manufacture a bi-layered tablet containing codeine phosphate in (at least) a first layer and an antihistamine and/or a decongestant and/or an expectorant in (at least) a second layer. Non-limiting examples of possible active ingredients (in addition to the antitussive morphine derivative) in an exemplary range as described in the following Table 1 can be employed depending on the specific therapeutic effect desired.

TABLE 1

| | | | Preferred | | OTC |
|---|---|---|---|---|---|
| | Amount per | | Amount | | Daily |
| Active ingredient | Tablet | | per Tablet | | Dosage |
| ANTIHISTAMINES | | | | | |
| Azelastine hydrochloride | 0.1-2.0 | mg | 0.125 | mg | |
| Azatadine hydrochloride | 0.1-4.0 | mg | 1 | mg | |
| Brompheniramine maleate | 0.1-64 | mg | 2-16 | mg | 24 mg |
| Dexbrompheniramine | 0.1-24 | mg | 3-6 | mg | 12 mg |
| maleate | | | | | |
| Carbinoxamine maleate | 0.1-16 | mg | 4 | mg | |
| Cetirizine hydrochloride | 0.1-40 | mg | 5-10 | mg | |
| Chlorcyclizine | 0.1-300 | mg | | | 75 mg |
| Chlorpheniramine maleate | 0.1-64 | mg | 2-16 | mg | 24 mg |
| Chlorpheniramine polistirex | 0.1-32 | mg | 4-8 | mg | |
| Clemastine | 0.1-12 | mg | 0.5-2.68 | mg | |
| Cyproheptadine | 0.1-16 | mg | 2-4 | mg | |
| Dexchlorpheniramine | 0.1-24 | mg | 2 | mg | 12 mg |
| maleate | | | | | |
| Cyproheptadine | 0.1-32 | mg | 2-4 | mg | |
| hydrochloride | | | | | |
| Diphenhydramine | 0.1-300 | mg | 10-50 | mg | 300 mg |
| hydrochloride | | | | | |
| Diphenhydramine citrate | 0.1-2000 | mg | | | 456 mg |
| Bromodiphenhydramine | 0.1-200 | mg | 12.5-25 | mg | |
| hydrochloride | | | | | |
| Doxylamine succinate | 0.1-200 | mg | 12.5-25 | mg | 75 mg |
| Fexofenadine hydrochloride | 0.1-720 | mg | 30-180 | mg | |
| Hydroxyzine hydrochloride | 0.1-400 | mg | 10-100 | mg | |
| Hydroxyzine pamoate | 0.1-400 | mg | 25-100 | mg | |
| Loratadine | 0.1-80 | mg | 1-10 | mg | |
| Desloratadine | 0.1-40 | mg | 5 | mg | |
| Phenindamine tartrate | 0.1-750 | mg | | | 150 mg |
| Pheniramine maleate | 0.1-750 | mg | | | 150 mg |
| Pyrilamine maleate | 0.1-200 | mg | 25 | mg | 200 mg |
| Terfenadine | | | | | |
| Thenyldiamine | | | | | |
| Thonzylamine | 0.1-3000 | mg | | | 600 mg |
| Thymol | | | | | |
| Tripelennamine | 0.1-400 | mg | 25-50 | mg | |
| hydrochloride | | | | | |
| Triprolidine hydrochloride | 0.1-40 | mg | 1.25-5 | mg | 10 mg |
| EXPECTORANT | | | | | |
| Guaifenesin | 0.1-2000 | mg | 50-1200 | | 2400 mg |

### EXAMPLE 16

### Bi-Layered Tablet (Direct Compression and Wet Granulation)

A bi-layered tablet in accordance with the present invention which contains codeine phosphate in both an immediate release layer and a sustained release layer is illustrated as follows:

| | | |
|---|---|---|
| | Weight/tablet | Weight/1 kg |
| Ingredients | (mgs) | batch (gms) |

Layer 1 (Immediate release)

| | | |
|---|---|---|
| Codeine Phosphate | 15.0 | 46.2 |
| Silicified Microcrystalline Cellulose | 73.5 | 226.4 |
| Croscarmellose Sodium | 10.0 | 30.8 |
| Magnesium Stearate | 1.5 | 4.6 |

Layer 2 (Sustained release)

| | | |
|---|---|---|
| Codeine Phosphate | 45.0 | 138.6 |
| Microcrystalline Cellulose (PH 102) | 20.0 | 61.6 |
| Povidone | 8.0 | 24.6 |
| Methocel K4M Premium | 150.0 | 462.0 |
| Magnesium Stearate | 2.0 | 6.2 |
| Total | 325.0 | 1000.0 |

Procedure:

(a) Immediate release layer #1: Mix the prescreened (# 30 mesh) codeine phosphate, silicified microcrystalline cellulose and croscarmellose sodium, in a V shaped blender for 20 minutes. Add prescreened magnesium stearate in a V shaped blender and mix for 3 minutes.

(b) Sustained release layer #2: Mix the codeine phosphate, Methocel K4M Premium and microcrystalline cellulose in a high shear mixer/granulator for 10 minutes. Granulate the above blend using a 30% povidone solution (8.0 gms povidone in 26.7 gms purified water). Dry the granulation until the LOD is less than 2.0%. Screen granules through a USP sieve size # 14. Add the granules and the prescreened magnesium stearate in a V shaped blender and mix for 3 minutes.

Manufacture bi-layered tablets using a rotary bi-layer tablet press where in each tablet layer #1 is 100 mgs and layer #2 is 225 mgs.

### EXAMPLE 17

### Single Layer Tablet or Capsule

A single layer tablet or a capsule in accordance with the present invention which contains codeine phosphate both in an immediate release form and in a sustained release form is illustrated as follows:

| Ingredients | Amount (mg)/tablet |
|---|---|
| Codeine Phosphate Ion- | Equivalent to 45 mgs |
| Exchange Complex | of Codeine Phosphate |
| Codeine Phosphate | 15 |
| Eudragit ® L 100 | 10 to 100 |
| Microcrystalline Cellulose | q.s* |
| Magnesium Stearate | 5 |
| Total | 500 |

*Added to make remainder of weight.

The formula described above serves as a non-limiting example. Any active drug which is in the form of a salt, such as codeine, or dihydrocodeine, or hydrocodone can be incorporated as an ion-exchange resin complex.

Procedure:

(1) Add the appropriate amount of sodium polystyrene sulphonate USP (e.g. Amberlite® IRP 69) to a codeine phosphate solution.

(2) Stir the mix for 12 hrs to allow complete drug/resin complex formation.

(3) Separate and dry the insoluble drug/resin complex.

(4) Granulate the drug/resin complex with a delayed release/enteric polymer (e.g. Eudragit® L 100, Kollidon® MAE, Aquacoat® cPD) and dry the granules.

(5) Mill the granules, if needed.

(6) To the milled granules add the appropriate amount of microcrystalline cellulose and the remaining codeine phosphate in a V shaped blender and mix for 15 minutes.

(7) Add prescreened (sieve # 30) magnesium stearate to the above blend and mix for 3 minutes.

(8) Fill into appropriate capsules.

### EXAMPLE 18

### Extended Release Suspension (Gel)

An extended release suspension (in the form of a gel) in accordance with the present invention which contains a codeine phosphate ion-exchange complex and promethazine hydrochloride is illustrated as follows (note that the codeine phosphate is used in a controlled release form since it has a shorter half-life than the promethazine hydrochloride):

| Ingredients | Amount/5 ml | |
|---|---|---|
| Codeine Phosphate Ion- | Equivalent to 30 mgs | |
| Exchange Complex | of Codeine Phosphate | |
| Promethazine HCl | 25 | mgs |
| Eudragit ® L 100 | 0.2 to 2.8 | grams |
| Glycerin | 315 | mgs |
| Polysorbate 80 | 1.5 | mgs |
| Carbomer (e.g., Carbopol ® 974) | 37.5 | mgs |
| Methyl Paraben | 9 | mgs |
| Propyl Paraben | 1 | mgs |
| Saccharin Sodium cryst., USP | 0.1 | mg |
| Artificial Grape Flavor | 5 | mgs |
| FD&C Red # 40 Dye | 0.5 | mgs |

| | |
|---|---|
| Sodium Hydroxide | q.s. |
| Water | q.s |

Procedure:

(1) Add the appropriate amount of sodium polystyrene sulphonate USP (e.g. Amberlite® IRP 69) to a codeine phosphate solution.

(2) Stir the mix for 12 hrs to allow complete drug/resin complex formation.

(3) Separate and dry the insoluble drug/resin complex.

(4) Granulate the drug/resin complex with a delayed release/enteric polymer (e.g. Eudragit® L 100, Kollidon® MAE, Aquacoat® cPD) and dry the granules.

(5) Mill the granules, if needed.

(6) To an appropriate amount of water add the following ingredients and dissolve: promethazine hydrochloride, Carbomer (e.g., Carbopol® 974), glycerin, polysorbate 80, methyl paraben, propyl paraben, artificial grape flavor, FD&C red # 40 dye.

(7) Add milled granules.

(8) Add water to 95% of final volume.

(9) Agitate at suitable rate to avoid settling of the suspension and maintain a homogeneous product mixture.

(10) Neutralize the solution to form a gel using a IN sodium hydroxide solution. Add water to make final volume.

(11) Fill in suitable containers ensuring that the product is homogeneous throughout the filling operation.

### EXAMPLE 19

### Extended Release Suspension (Liquid)

An extended release suspension (in the form of a liquid) in accordance with the present invention which contains a codeine phosphate ion-exchange complex and promethazine hydrochloride is illustrated as follows:

| Ingredients | Amount/5 ml | |
|---|---|---|
| Codeine Phosphate Ion- | Equivalent to 45 mgs | |
| Exchange Complex | of Codeine Phosphate | |
| Promethazine HCl | 25 | mgs |
| Eudragit ® L 100 | 0.2 to 2.8 | grams |
| Silica, colloidal anhydrous, NF | 100 | mgs |
| Glycerin | 740 | mgs |
| Xylitol, NF | 800 | mgs |
| Sodium Citrate, USP | 100 | mgs |
| Saccharin Sodium cryst., USP, | 0.1 | mg |
| Sodium Benzoate | 7.5 | mgs |
| Citric Acid Monohydrate, USP | 8.0 | mgs |
| Artificial Grape Flavor | 5 | mgs |
| FD&C Red # 40 Dye | 0.5 | mgs |

| | |
|---|---|
| Water | q.s |

Manufacturing Process for 1000 L Batch:

Add the appropriate amount of sodium polystyrene sulphonate USP (e.g. Amberlite® IRP 69) to a codeine phosphate solution. Stir the mix for 12 hrs to allow complete drug/resin complex formation. Separate and dry the insoluble drug/resin complex. Granulate the drug/resin complex with a delayed release/enteric polymer (e.g. Eudragit® L 100, Kollidon® MAE, Aquacoat® CPD) and dry the granules. Mill the granules, if needed.

In a suitably sized stainless steel vessel, dissolve saccharin sodium, sodium benzoate, citric acid, and sodium citrate in approximately 50 L of warm (about 45° C.), purified water. In another large stainless steel drum mix the silica, codeine phosphate ion-exchange complex, and promethazine hydrochloride until a uniform and consistent mixture is obtained. In a separate 1000 L stainless steel tank equipped with a suitably sized homogenizer/disperser add about 100 L of purified water. With the homogenizer on, add the silica mixture containing codeine phosphate ion-exchange complex, and promethazine hydrochloride. Add the previously prepared solution of saccharin sodium, sodium benzoate, citric acid, and sodium citrate to the 1000 L tank. Rinse the first vessel with about 2 L of water and transfer the rinsate to the 1000 L tank. Add the remaining ingredients and homogenize for 15 minutes.

### EXAMPLE 20

### Extended Release Suspension (Liquid)

An extended release suspension (in the form of a liquid) in accordance with the present invention which contains a codeine phosphate ion-exchange complex, pseudoephedrine tannate and chlorpheniramine tannate is illustrated as follows:

| Ingredients | Amount/5 ml | |
|---|---|---|
| Codeine Phosphate Ion-Exchange | Equivalent to 45 mgs of Codeine | |
| Complex | Phosphate | |
| Pseudoephedrine Tannate | 75.0 | |
| Chlorpheniramine Tannate | 4.5 | |
| Eudragit ® L 100 | 0.2 to 2.8 | grams |
| Silica, colloidal anhydrous, NF | 100 | mgs |
| Glycerin | 740 | mgs |
| Xylitol, NF | 800 | mgs |
| Sodium Citrate, USP | 100 | mgs |
| Saccharin Sodium cryst., USP, | 0.1 | mg |
| Sodium Benzoate | 7.5 | mgs |
| Citric Acid Monohydrate, USP | 8.0 | mgs |
| Artificial Grape Flavor | 5 | mgs |
| FD&C Red # 40 Dye | 0.5 | mgs |

| | |
|---|---|
| Water | q.s |

Manufacturing Process for 1000 kg Batch:

Add the appropriate amount of sodium polystyrene sulphonate USP (e.g. Amberlite® IRP 69) to a codeine phosphate solution. Stir the mix for 12 hrs to allow complete drug/resin complex formation. Separate and dry the insoluble drug/resin complex. Granulate the drug/resin complex with a delayed release/enteric polymer (e.g. Eudragit® L 100, Kollidon® MAE, Aquacoatg CPD) and dry the granules. Mill the granules, if needed.

In a suitably sized stainless steel vessel, dissolve saccharin sodium, sodium benzoate, citric acid, and sodium citrate in approximately 50 L of warm (about 45° C.), purified water. In another large stainless steel drum mix the silica, codeine phosphate ion-exchange complex, pseudoephedrine tannate, and the chlorpheniramine tannate until a uniform and consistent mixture is obtained. In a separate 1000 L stainless steel tank equipped with a suitably sized homogenizer/disperser add about 100 L of purified water. With the homogenizer on, add the silica mixture containing codeine phosphate ion-exchange complex, pseudoephedrine tannate, and the chlorpheniramine tannate. Add the previously prepared solution of saccharin sodium, sodium benzoate, citric acid, and sodium citrate to the 1000 L tank. Rinse the first vessel with about 2 L of water and transfer the rinsate to the 1000 L tank. Add the remaining ingredients and homogenize for 15 minutes.

### REFERENCE EXAMPLE 1

### Extended Release Suspension

An extended release suspension which contains a hydrocodone bitartrate ion-exchange complex, a dexchlorpheniramine maleate ion-exchange complex and a phenylepherine hydrochloride ion-exchange complex is illustrated as follows:

| Ingredients | Amount/5 ml | |
|---|---|---|
| Hydrocodone Bitartrate Ion-Exchange | Equivalent to 8 mgs of | |
| Complex | Hydrocodone bitartarate | |
| Dexchlorpheniramine Maleate Ion- | Equivalent to 4 mgs of | |
| Exchange Complex | Dexchlorpheniramine Maleate | |
| Phenylepherine HCl Ion-Exchange | Equivalent to 10 mgs of | |
| Complex | Phenylepherine HCl | |
| Eudragit ® L 100 | 0.2 to 2.8 | grams |
| Glycerin | 315 | mgs |
| Polysorbate 80 | 1.5 | mgs |
| Carbomer (e.g., Carbopol ® 974) | 15 | mgs |
| Methyl Paraben | 9 | mgs |
| Propyl Paraben | 1 | mgs |
| Artificial Grape Flavor | 5 | mgs |
| FD&C Red # 40 Dye | 0.5 | mgs |

| | |
|---|---|
| Water | q.s |

The formula described above serves as a non-limiting example. Any active drug which is in the form of a salt, such as codeine, or dihydrocodeine, or hydrocodone can be incorporated as an ion-exchange resin complex.

Procedure:

(1) Add the appropriate amount of sodium polystyrene sulphonate USP (e.g. Amberlite® IRP 69) to a codeine phosphate, dexchlorpheniramine maleate and phenylepherine HCl solution.

(2) Stir the mix for 12 hrs to allow complete drug/resin complex formation.

(3) Separate and dry the insoluble drug/resin complex.

(4) Granulate the drug/resin complex with a delayed release/enteric polymer (e.g. Eugragit® 100, Kollidon® MAE, Aquacoa®t cPD) and dry the granules.

(5) Mill the granules, if needed.

(6) To an appropriate amount of water add the following ingredients and dissolve: Carbomer (e.g., Carbopol® 974), glycerin, polysorbate 80, methyl paraben, propyl paraben, artificial grape flavor, FD&C red # 40 dye.

(7) Add milled granules.

(8) Add water to make up to a final volume.

(9) Agitate at suitable rate to avoid settling of the suspension and maintain a homogeneous product mixture.

(10) Fill in suitable containers ensuring that the product is homogeneous throughout the filling operation.

### REFERENCE EXAMPLE 2

### Suspension Formula

A suspension formula which comprises codeine phosphate and phenylepherine tannate is illustrated as follows:

| | g/100 mL = | | kg/batch = | |
|---|---|---|---|---|
| Ingredients | 120 g | | 1000 kg | |
| Codeine Phosphate | 0.500 | | 4.167 | |
| Phenylepherine Tannate | 0.800 | | 6.667 | |
| Silica, colloidal anhydrous, NF | 1.73 | | 14.417 | |
| Hydroxyethylcellulose, NF | 0.05 | | 0.417 | |
| Sorbitol Solution 70% (non-crystallizing), | 34.00 | | 283.333 | |
| NF | | | | |
| Glycerol | 14.75 | | 122.917 | |
| Xylitol, NF | 16.00 | | 133.333 | |
| Sodium Citrate, USP | 2.00 | | 16.667 | |
| Saccharin Sodium cryst., USP, | 0.01 | | 0.083 | |
| Sodium Benzoate, NF | 0.15 | | 1.250 | |
| Citric Acid Monohydrate, USP | 0.16 | | 1.333 | |
| Strawberry Flavor | 0.15 | | 1.250 | |
| Banana Flavor | 0.15 | | 1.250 | |
| Purified Water | 49.55 | | 412.917 | |
| Total Amount | 120.000 | g | 1000.000 | kg |

Manufacturing Process for 1000 kg Batch:

In a suitably sized stainless steel vessel, dissolve saccharin sodium, sodium benzoate, citric acid, and sodium citrate in approximately 50 L of warm (about 45° C.), purified water. In another large stainless steel drum mix the silica, codeine phosphate, and micronized phenylepherine tannate until a uniform and consistent mixture is obtained. In a separate 1000 L stainless steel tank equipped with a suitably sized homogenizer/disperser add about 100 L of purified water. With the homogenizer on, add the silica mixture containing phenylepherine tannate and codeine phosphate. Add the previously prepared solution of saccharin sodium, sodium benzoate, citric acid, and sodium citrate to the 1000 L tank. Rinse the first vessel with about 2 L of water and transfer the rinsate to the 1000 L tank. Add the remaining ingredients and homogenize for 15 minutes. Filter product through a 10 micron filter and fill in appropriately sized containers.

To make products with other agents such as antihistamines, decongestants, or expectorants, one or more combinations of each of the ingredients in a range as described in Table 1 below can be made depending on the specific therapeutic effect desired.

### REFERENCE EXAMPLE 3

### Liquid Formula

A liquid dosage form which comprises codeine phosphate and phenylepherine hydrochloride is illustrated as follows:

| Ingredients | Per 5 mL | Per 425 L |
|---|---|---|
| Codeine Phosphate USP | 30 mg | 2.550 kg |
| Phenylepherine Hydrochloride USP | 10.0 mg | 0.850 kg |
| Methyl Paraben USP | 9.0 mg | 0.765 kg |
| Propyl Paraben USP | 1.0 mg | 0.085 kg |
| Propylene Glycol USP | 259 mg | 22.016 kg |
| Saccharin Sodium USP | 3.18 mg | 0.270 kg |
| Citric Acid USP | 5.0 mg | 0.425 kg |
| Strawberry Flavor | 10 mg | 0.850 kg |
| Banana Flavor | 10 mg | 0.850 kg |
| Sorbitol Solution 70% USP | 3212.5 mg | 273.1 kg |
| Purified Water, as required to q.s. to | 5.0 mL | 425 L |

Manufacturing Process for 425 L Batch Size:

In a suitably sized stainless steel vessel, dissolve methyl paraben and propyl paraben in approximately 50 L of warm (about 45° C.), purified water. Add about half of the propylene glycol and mix for about 1 hr. In a separate 1000 L stainless steel tank equipped with a suitably sized agitator, add about 50 L of purified water. With the agitator on, add phenylepherine hydrochloride, codeine phosphate, saccharin sodium and citric acid and dissolve. Add the previously prepared paraben/propylene glycol solution to the 1000 L tank. Rinse the first vessel with about 2 L of water and transfer the rinsate to the 1000 L tank. Add the remaining propylene glycol to a suitably sized stainless steel vessel and dissolve the strawberry and banana flavors. Transfer this to the 1000 L tank. Rinse the container with 2 L of purified water and transfer to the 1000 L tank. With the agitator on, add the sorbitol solution 70% to the 1000 L tank. In a suitably sized stainless steel vessel, dissolve the codeine phosphate in about 5 L of purified water and transfer to the 1000 L tank. Rinse the container with about 2 L of purified water and transfer to the 1000 L tank. Stop the agitator and let the solution stand for 15 minutes. QS to 425 L with purified water. Filter product through a 1 micron filter and fill in appropriately sized containers.

To make products with other antihistamines, decongestants, or expectorants, one or more combinations of each of the ingredients in a range as described in Table 1 above can be made depending on the specific therapeutic effect desired.

It is noted that the foregoing examples have been provided merely for the purpose of explanation and are in no way to be construed as limiting of the present invention. While the present invention has been described with reference to exemplary embodiments, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

1. A pharmaceutical dosage form which comprises (a) a first drug which comprises at least one morphine derivative having antitussive activity and (b) at least one second drug, wherein the dosage form provides a plasma concentration within a therapeutic range of the at least one second drug over a period which is coextensive with at least about 70% of a period over which the dosage form provides a plasma concentration within a therapeutic range of the first drug.

2. The dosage form of claim 1, wherein the at least one of morphine derivative comprises at least one of codeine, dihydrocodeine, hydrocodone and pharmaceutically acceptable salts thereof.

3. The dosage form of claim 2, wherein the first drug comprises at least one of codeine phosphate, dihydrocodeine bitartrate and hydrocodone bitartrate.

4. The dosage form of claim 2, wherein the first drug comprises codeine phosphate.

5. The dosage form of claim 2, wherein the at least one second drug comprises at least one of a decongestant, expectorant, mucus thinning drug, and antihistamine.

6. The dosage form of claim 1, wherein the at least one second drug comprises a decongestant.

7. The dosage form of claim 6, wherein the second drug comprises at least one of phenylepherine, pseudoephedrine and pharmaceutically acceptable salts thereof.

8. The dosage form of claim 2, wherein the at least one second drug comprises an antihistamine.

9. The dosage form of claim 8, wherein the antihistamine comprises at least one of chlorpheniramine, promethazine, carbinoxamine and pharmaceutically acceptable salts thereof.

10. The dosage form of claim 1, wherein the at least one second drug comprises an expectorant.

11. The dosage form of claim 10, wherein the expectorant comprises guaifenesin.

12. The dosage form of claim 5, wherein a plasma half-life of the at least one second drug differs from a plasma half-life of the first drug by at least about 2 hours.

13. The dosage form of claim 3, wherein a plasma half-life of the at least one second drug differs from a plasma half-life of the first drug by at least about 3 hours.

14. The dosage form of claim 1, wherein a plasma half-life of the at least one second drug differs from a plasma half-life of the first drug by at least about 4 hours.

15. The dosage form of claim 5, wherein the period of a plasma concentration within the therapeutic range of the at least one second drug is coextensive with at least about 80% of the period of a plasma concentration within the therapeutic range of the first drug.

16. The dosage form of claim 2, wherein the period of a plasma concentration within the therapeutic range of the at least one second drug is coextensive with at least about 90% of the period of a plasma concentration within the therapeutic range of the first drug.

17. The dosage form of claim 1, wherein the period of a plasma concentration within the therapeutic range of the at least one second drug is coextensive with at least about 95% of the period of a plasma concentration within the therapeutic range of the first drug.

18. The dosage form of claim 1, wherein the dosage form comprises a tablet.

19. The dosage form of claim 18, wherein the tablet comprises at least two layers.

20. The dosage form of claim 19, wherein the tablet is a bi-layered tablet.

21. The dosage form of claim 18, wherein the tablet comprises a matrix which comprises the first drug and has dispersed therein particles which comprise the at least one second drug.

22. The dosage form of claim 1, wherein the dosage form comprises one of a solution and a suspension.

23. A bi-layered tablet which comprises a first layer and a second layer, the first layer comprising a first drug which comprises at least one morphine derivative having antitussive activity, and the second layer comprising at least one second drug which is selected from decongestants, expectorants, mucus thinning drugs, and antihistamines, wherein the bi-layered tablet provides a plasma concentration within a therapeutic range of the at least one second drug over a period which is coextensive with at least about 70% of a period over which the bi-layered tablet provides a plasma concentration within a therapeutic range of the first drug.

24. The bi-layered tablet of claim 23, wherein the first layer comprises at least one of codeine, dihydrocodeine, hydrocodone and pharmaceutically acceptable salts thereof.

25. The bi-layered tablet of claim 24, wherein the second layer comprises at least one of phenylepherine, pseudoephedrine, chlorpheniramine, carbinoxamine, promethazine, guaifenesin and pharmaceutically acceptable salts thereof.

26. The bi-layered tablet of claim 24, wherein the tablet comprises at least two of phenylepherine, pseudoephedrine, chlorpheniramine, carbinoxamine, promethazine, guaifenesin and pharmaceutically acceptable salts thereof.

27. The bi-layered tablet of claim 23, wherein the first layer only comprises one or more of codeine, dihydrocodeine, hydrocodone and pharmaceutically acceptable salts thereof as active ingredient(s).

28. The bi-layered tablet of claim 24, wherein the period of a plasma concentration within the therapeutic range of the at least one second drug is coextensive with at least about 80% of the period of a plasma concentration within the therapeutic range of the first drug.

29. The bi-layered tablet of claim 25, wherein the period of a plasma concentration within a therapeutic range of the at least one second drug is coextensive with at least about 90% of the period of a plasma concentration within a therapeutic range of the first drug.

30. The bi-layered tablet of claim 23, wherein at least one of the first and second layers is an immediate release layer.

31. The bi-layered tablet of claim 30, wherein the first layer is an immediate release layer.

32. The bi-layered tablet of claim 30, wherein the second layer is an immediate release layer.

33. The bi-layered tablet of claim 23, wherein both of the first and second layers are controlled release layers.

34. The bi-layered tablet of claim 24, wherein the first layer comprises a total of from about 0.1 mg to about 120 mg of at least one of codeine, dihydrocodeine, hydrocodone and pharmaceutically acceptable salts thereof.

35. The bi-layered tablet of claim 34, wherein the first layer comprises a total of from about 5 mg to about 90 mg of at least one of codeine, dihydrocodeine, hydrocodone and pharmaceutically acceptable salts thereof.

36. The bi-layered tablet of claim 35, wherein the first layer comprises a total of from about 25 mg to about 50 mg of at least one of codeine, dihydrocodeine, hydrocodone and pharmaceutically acceptable salts thereof.

37. The bi-layered tablet of claim 34, wherein the second layer comprises at least one of (i) from about 0.1 mg to about 16 mg of chlorpheniramine maleate or an equivalent amount of at least one other pharmaceutically acceptable salt of chlorpheniramine; (ii) from about 1 mg to about 90 mg of phenylepherine hydrochloride or an equivalent amount of at least one other pharmaceutically acceptable salt of phenylepherine; (iii) from about 1 mg to about 240 mg of pseudoephedrine hydrochloride or an equivalent amount of at least one other pharmaceutically acceptable salt of pseudoephedrine; (iv) from about 0.1 mg to about 75 mg of promethazine hydrochloride or an equivalent amount of at least one other pharmaceutically acceptable salt of promethazine; (v) from about 0.1 mg to about 32 mg of carbinoxamine maleate or an equivalent amount of at least one other pharmaceutically acceptable salt of carbinoxamine; and (vi) form about 1 mg to about 2400 mg of guaifenesin or an equivalent amount of at least one pharmaceutically acceptable salt of guaifenesin.

38. The bi-layered tablet of claim 35, wherein the first layer comprises at least one of (i) from about 1 mg to about 90 mg of phenylepherine hydrochloride or an equivalent amount of at least one other pharmaceutically acceptable salt of phenylepherine; and (ii) from about 1 mg to about 240 mg of pseudoephedrine hydrochloride or an equivalent amount of at least one other pharmaceutically acceptable salt of pseudoephedrine, and the second layer comprises at least one of an antihistamine and an expectorant.

39. A multi-layered tablet which comprises at least a first layer and a second layer, wherein the first layer comprises at least one of codeine, dihydrocodeine, hydrocodone and a pharmaceutically acceptable salt thereof and the second layer comprises at least one drug which is selected from decongestants, expectorants, mucus thinning drugs, analgesics and antihistamines.

40. The multi-layered tablet of claim 39, wherein the first layer is an immediate release layer.

41. The multi-layered tablet of claim 39, wherein the first layer is a controlled release layer.

42. The multi-layered tablet of claim 41, wherein the second layer is a controlled release layer.

43. The multi-layered tablet of claim 39, wherein the first layer comprises at least one of codeine phosphate, dihydrocodeine bitartrate and hydrocodone bitartrate.

44. The multi-layered tablet of claim 42, wherein the first layer does not contain any active ingredient which is different from codeine, dihydrocodeine, hydrocodone and pharmaceutically acceptable salts thereof.

45. The multi-layered tablet of claim 39, wherein the tablet comprises at least one of dextromethorphan, phenylepherine, pseudoephedrine, guaifenesin, chlorpheniramine, carbinoxamine, promethazine and pharmaceutically acceptable salts thereof.

46. The multi-layered tablet of claim 39, wherein the tablet comprises at least two of dextromethorphan, phenylepherine, pseudoephedrine, guaifenesin, chlorpheniramine, carbinoxamine, promethazine and pharmaceutically acceptable salts thereof.

47. The multi-layered tablet of claim 39, wherein the at least one drug in the second layer has a plasma half-life which differs by at least about 1 hour from the a plasma half-life of the at least one of codeine, dihydrocodeine, hydrocodone and pharmaceutically acceptable salts thereof.

48. The multi-layered tablet of claim 47, wherein the tablet provides a plasma concentration within a therapeutic range of the at least one drug in the second layer over a period which is coextensive with at least about 80% of a period over which the tablet provides a plasma concentration within a therapeutic range of the at least one of codeine, dihydrocodeine, hydrocodone and pharmaceutically acceptable salts thereof.

49. The multi-layered tablet of claim 48, wherein the at least one drug in the second layer comprises one or more of phenylepherine, pseudoephedrine, chlorpheniramine and pharmaceutically acceptable salts thereof.

50. The multi-layered tablet of claim 39, wherein the layers are discrete zones which are arranged adjacent to each other.

51. The multi-layered tablet of claim 39, wherein the second layer is partially or completely surrounded by the first layer.

52. The multi-layered tablet of claim 39, wherein the second layer is coated with the first layer.

53. A liquid dosage form which comprises (a) at least one of codeine, dihydrocodeine, hydrocodone and pharmaceutically acceptable salts thereof and (b) at least one drug which is selected from decongestants, expectorants, mucus thinning drugs, and antihistamines, wherein the liquid dosage form provides a plasma concentration within a therapeutic range of (b) over a period which is coextensive with at least about 70% of a period over which the liquid dosage form provides a plasma concentration within a therapeutic range of (a).

54. The liquid dosage form of claim 53, wherein the liquid dosage form comprises a suspension.

55. The liquid dosage form of claim 54, wherein the suspension comprises a gel.

56. The liquid dosage form of claim 53, wherein at least a part of (b) is present as a complex with a complexing agent.

57. The liquid dosage form of claim 53, wherein at least a part of (a) is present as a complex with a complexing agent.

58. The liquid dosage form of claim 56, wherein at least a part of (a) is present as a complex with a complexing agent.

59. The liquid dosage form of claim 57, wherein the complexing agent comprises an ion-exchange resin.

60. The liquid dosage form of claim 59, wherein the ion-exchange resin comprises sodium polystyrene sulfonate.

61. The liquid dosage form of claim 54, wherein the suspension comprises particles of a complex of at least a part of component (b) with an ion-exchange resin, which particles are provided, at least in part, with a controlled release coating.

62. The liquid dosage form of claim 61, wherein the controlled release coating comprises an organic polymer.

63. The liquid dosage form of claim 62, wherein the organic polymer comprises a polyacrylate.

64. A method of concurrently alleviating a condition which can be alleviated by administering codeine, dihydrocodeine, or hydrocodone and at least one other condition which can be alleviated by administering a drug which is at least one of a decongestant, expectorant, mucus thinning drug, and antihistamine, wherein the method comprises administering the pharmaceutical dosage form of claim 1 to a subject in need thereof.

65. A method of concurrently alleviating a condition which can be alleviated by administering codeine, dihydrocodeine, or hydrocodone and at least one other condition which can be alleviated by administering a drug which is at least one of a decongestant, expectorant, mucus thinning drug, and antihistamine, wherein the method comprises administering the multi-layered tablet of claim 39 to a subject in need thereof.

66. The method of claim 65, wherein the condition which can be alleviated by administering codeine, dihydrocodeine, or hydrocodone comprises coughing.

67. The method of claim 64, wherein the dosage form is administered not more than about three times per day.

68. The method of claim 67, wherein the multi-layered tablet is administered not more than about twice per day.

69. A method of concurrently alleviating a condition which can be alleviated by administering codeine, dihydrocodeine, or hydrocodone and at least one other condition which can be alleviated by administering a drug which is at least one of a decongestant, expectorant, mucus thinning drug, and antihistamine, wherein the method comprises administering the liquid dosage form of claim 53 to a subject in need thereof.

70. A process of making the pharmaceutical dosage form of claim 1, wherein the method comprises preparing a first composition which comprises the first drug and a second composition which comprises the at least one second drug, and combining the first and the second compositions to form the dosage form.

71. The process of claim 70, wherein the first and second compositions are combined by using a tablet press.

72. A pharmaceutical dosage form which comprises (a) a first drug which comprises at least one of codeine, dihydrocodeine, hydrocodone and pharmaceutically acceptable salts thereof and has a first plasma half-life and (b) at least one second drug which is selected from decongestants, expectorants, mucus thinning drugs, and antihistamines and has a second plasma half-life which differs from the first plasma half-life by at least about 2 hours, wherein the dosage form provides a plasma concentration within a therapeutic range of the at least one second drug over a period which is coextensive with at least about 80% of a period over which the dosage form provides a plasma concentration within a therapeutic range of the first drug.

73. The dosage form of claim 72, wherein the first plasma half-life differs by at least about 3 hours from the second plasma half-life.

74. The dosage form of claim 72, wherein the period of a plasma concentration within the therapeutic range of the at least one second drug is coextensive with at least about 90% of the period over which the dosage form provides a plasma concentration within the therapeutic range of the first drug.

75. The dosage form of claim 74, wherein the dosage form comprises a multi-layered tablet.

76. The dosage form of claim 72, wherein the dosage form is associated with instructions to administer the dosage form three or fewer times per day.

77. The dosage form of claim 75, wherein the dosage form is associated with instructions to administer the dosage form once or twice per day.

78. A pharmaceutical dosage form which comprises (a) at least one first morphine derivative in a first form or layer and (b) at least one second morphine derivative in a second form or layer which is different from the first form or layer, wherein the dosage form releases the at least one first morphine derivative at least one of over a different period and at a different rate than the at least one second morphine derivative.

79. The dosage form of claim 78, wherein the at least one first morphine derivative and the at least one second morphine derivative are the same.

80. The dosage form of claim 78, wherein the at least one first morphine derivative and the at least one second morphine derivative are independently selected from codeine, dihydrocodeine, hydrocodone and pharmaceutically acceptable salts thereof.

81. The dosage form of claim 79, wherein the at least one first morphine derivative and the at least one second morphine derivative comprise at least one of codeine phosphate, dihydrocodeine bitartrate and hydrocodone bitartrate.

82. The dosage form of claim 78, wherein the dosage form comprises codeine phosphate.

83. The dosage form of claim 80, wherein the first form or layer is an immediate release form or layer and the second form or layer is a controlled release form or layer.

84. The dosage form of claim 78, wherein the dosage form is a multi-layered tablet which comprises at least one immediate release layer and at least one controlled release layer which independently comprise at least one of codeine, dihydrocodeine, hydrocodone and pharmaceutically acceptable salts thereof.

85. The dosage form of claim 84, wherein the dosage form further comprises at least one additional drug which is selected from decongestants, expectorants, mucus thinning drugs, and antihistamines.

86. The dosage form of claim 85, wherein at least the immediate release layer thereof comprises the at least one additional drug.

87. The dosage form of claim 85, wherein at least the controlled release layer thereof comprises the at least one additional drug.

88. The dosage form of claim 78, wherein the dosage form is a liquid which comprises the at least one first morphine derivative in a free form and the at least one second morphine derivative as a complex with a complexing agent.

89. The dosage form of claim 88, wherein the at least one first morphine derivative and the at least one second morphine derivative are the same.

90. The dosage form of claim 89, wherein the complexing agent comprises an ion-exchange resin.

91. The dosage form of claim 88, wherein the liquid comprises a suspension.

92. The dosage form of claim 78, wherein the dosage form releases the at least one first morphine derivative over a different period and at a different rate than the at least one second morphine derivative.

93. The dosage form of claim 78, wherein the dosage form releases the at least one first morphine derivative over a different period than the at least second morphine derivative.

94. The dosage form of claim 93, wherein the dosage form releases the at least one first morphine derivative over a first period and the at least one second morphine derivative over a second period and not more than about 30% of the second period are coextensive with all or a part of the first period.

95. The dosage form of claim 94, wherein there is substantially no overlap between the first and second periods.

96. The dosage form of claim 78, wherein the dosage form releases the at least one first morphine derivative at a different rate than the at least second morphine derivative.

97. The dosage form of claim 79, wherein not more than about 30% of a period over which a plasma concentration within a therapeutic range of the morphine derivative is provided by (b) is coextensive with all or a part of a period over which (a) provides a plasma concentration within the therapeutic range, provided that the plasma concentrations provided by (a) and (b) together at any time following ingestion of the dosage form are not higher than a maximum plasma concentration of the therapeutic range of the morphine derivative.

98. The dosage form of claim 97, wherein not more than about 10% of the period over which a plasma concentration within the therapeutic range is provided by (b) is coextensive with all or a part of the period over which (a) provides a plasma concentration within the therapeutic range.
